(19) United States
(12) Patent Application Publication
(10) Pub. No.: US 2023/0399418 A1
(43) Pub. Date: Dec. 14, 2023

(54) ANTIBODIES FOR OPIOID TREATMENTS

(71) Applicant: THE SCRIPPS RESEARCH INSTITUTE, La Jolla, CA (US)

(72) Inventors: Kim D. Janda, La Jolla, CA (US); Paul T. Bremer, San Diego, CA (US); Bin Zhou, San Diego, CA (US)

(21) Appl. No.: 18/250,230

(22) PCT Filed: Nov. 8, 2021
(86) PCT No.: PCT/US21/58459

§ 371 Date: Apr. 24, 2023

Related U.S. Application Data
(60) Provisional application No. 63/111,699, filed on Nov. 10, 2020.

(51) Int. Cl. C07K 16/44; A61P 25/36

(52) U.S. Cl. CPC C07K 16/44; A61P 25/36; C07K 2317/622; C07K 2317/24

(57) ABSTRACT

The disclosure provides, inter alia, antibodies, pharmaceutical compositions containing antibodies, and the use of the antibodies and pharmaceutical compositions to treat or prevent opioid overdoses, to treat opioid use disorder, and to treat or prevent opioid-induced respiratory depression.

### CROSS-REFERENCE TO RELATED APPLICATIONS

This application claims priority to U.S. Application No. 63/111,699 (filed Nov. 10, 2020; now pending), the disclosure of which is incorporated by reference in its entirety.

### STATEMENT CONCERNING GOVERNMENT SUPPORT

This invention was made with government support under Grant No. DA046323 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 2014_1PC_20211105_SeqListing, created Nov. 5, 2021, 75,558 bytes, machine format IBM-PC, MS Windows operating system, is hereby incorporated by reference.

### BACKGROUND

The ongoing rise in opioid abuse creates a dire need for fast-acting, effective therapies to combat opioid intoxication and overdose. In early August 2017, the opioid crisis was declared a national emergency in the United States due to the ever-increasing number of opioid overdose reports. The etiology of ballooning opioid-related fatalities is multifactorial and most commonly involves the abuse of powerful semisynthetic and fully synthetic opioids, i.e., heroin and fentanyl, respectively. The fentanyl class of drugs are potent mu-opioid receptor agonists, and this pharmacological property is responsible for their pain relieving, addictive, and potentially dangerous central nervous system (CNS)-depressive effects. First discovered in 1960 by Paul Janssen, fentanyl is a Drug Enforcement Administration (DEA) schedule II drug that is routinely used in the clinic. Fentanyl can be administered intravenously for anesthesia, as well as transdermally and transmucosally via patches and lozenges, respectively, for mitigating pain in a variety of scenarios (e.g., postoperative, cancer-related and acute pain). However, fentanyl mirrors the abuse liability profile of heroin, and intentional abuse of prescription fentanyl patches has been reported. While heroin abuse has remained a public health concern for many decades, more recently, fentanyl poses even greater danger to illicit opioid users due to its enhanced potency.

The rise in opioid misuse has intensified the need for new and creative ways to both treat and counter their effects. Naloxone is effective in reversing heroin and prescription opioid-related overdose, but appears to be less effective against fentanyl and carfentanil. Specifically, the high potency of the fentanyl class of drugs, especially carfentanil, is reported to overwhelm the naloxone duration of action and therapeutic efficacy of typical naloxone doses. Fentanyl, while effective in pain medication and in anesthetic induction, is particularly dangerous when used illicitly due to its potency in respiratory depression. While the pharmacology of carfentanil is not well characterized in humans, it has similar properties to fentanyl but with greatly increased potency and a potential to cause “renarcotization” whereby a subject experiences opioid intoxication long after apparent recovery. Although this phenomenon has not been studied in humans, it is known to occur in large animals tranquilized with carfentanil, and administration of naloxone doses of 100-fold greater than the carfentanil doses are required to prevent renarcotization. In a clinical setting, continuous or multiple naloxone doses over time is required to sustain the reversal of fentanyl intoxication and to prevent possible renarcotization. Nalmefene is another opioid antagonist that has shown enhanced efficacy and duration of action against fentanyl and carfentanil-induced respiratory depression compared to naloxone. While opioid antagonists are useful in combatting opioid overdose, new drugs and new approaches are needed in the art to treat or prevent opioid overdose and to treat opioid use disorder. The disclosure is directed to this, as well as other, important ends.

### BRIEF SUMMARY

Provided herein are antibodies with high affinity and specificity to synthetic opioids, such as fentanyl, carfentanil, and analogues thereof. The disclosure provides antibodies comprising: (i) a light chain variable region which comprises a CDR L1 as set forth in SEQ ID NO:1, a CDR L2 as set forth in SEQ ID NO:2, and a CDR L3 as set forth in SEQ ID NO:3; and (ii) a heavy chain variable region which comprises a CDR H1 as set forth in SEQ ID NO:4, a CDR H2 as set forth in SEQ ID NO:5, and a CDR H3 as set forth in SEQ ID NO:6. The disclosure provides methods of preventing an opioid overdose, methods of treating an opioid overdose, and methods of treating opioid use disorder by administering to a patient an effective amount of any of the antibodies described herein. These and other embodiments of the disclosure are provided in detail herein.

### DESCRIPTION OF THE DRAWINGS

FIGS. 1A-1C show the efficacy and half-life determination of monoclonal antibodies (mAbs) in monkeys. FIG. 1A: rhesus monkeys (n=3) were given an IV dose of fentanyl followed by an 8 mg/kg IV dose of JBZ-1 or comparative mAb. The effects on tail withdrawal antinociception were observed and shown as a percentage of the maximum possible effect (MPE). FIG. 1B: cumulative dose fentanyl ED_{50S} were determined in the same monkeys at three time points post mAb infusion. FIG. 1C: blood samples were taken from the monkeys and analyzed by ELISA to determine antibody concentrations at the indicated time points.

FIGS. 2A-2C show the fentanyl pharmacokinetics in human mAb treated mice. Anti-fentanyl mAbs bind large quantities of drug in the blood to reduce peripheral drug exposure to the brain. FIG. 2A: time course experiment of blood fentanyl levels in mice (n=4) infused with mg/kg mAb, 48 h prior to IV 0.1 mg/kg fentanyl injection. FIG. 2B: fentanyl area under curve values derived from panel A data. FIG. 2C: fentanyl blood levels in mice treated with JBZ-1, JBZ-2, and P2B5 at 15 min and 3 h.

FIGS. 3A-3D show in vivo mAb-mediated antagonism of carfentanil-induced antinociception. FIGS. 3A-3B: JBZ-1 treatment 24-h prior to drug challenge significantly shifts carfentanil dose-effect curves rightward in tail flick and hot plate tests. FIG. 3C: comparison of fentanyl ED_{50S} in mice treated with JBZ-1, JBZ-2, and P2B5 in tail flick antinociception assay, showing superiority of JBZ-2. FIG. 3D: Comparison of the percentage of mice below the maximum possible effect cutoff in the hot plate antinociception assay when treated with JBZ-1, JBZ-2, and JBZ-3 showing superiority of JBZ-2.

FIGS. 4A-4C provide an evaluation of JBZ-2, JBZ-3, JBZ-4, and P2B5 antibodies in fentanyl antinociception. FIG. 4A: mAb (45 mg/kg) was given IP to n=4 Swiss Webster females and antinociception induced by 0.4 mg/kg IP fentanyl administered 4 h post-mAb. FIG. 4B: antinociception was followed for a 15-115 minute interval post-fentanyl and expressed as an average. FIG. 4C: fentanyl antinociception was retested in the same mice at 2 days post-mAb.

FIGS. 5A-5B show quantification of antibody and fentanyl in peripheral mouse blood. FIG. 5A: the same mice from the antinociception study were administered an IP fentanyl challenge (0.4 mg/kg), and blood samples were taken 20 min after drug injection. Samples were basified, combined with deuterated internal standard, extracted with organic solvent and analyzed by LC-MS/MS using a standard curve to interpolate unknown concentrations. FIG. blood samples taken before drug administration at 2 d post-mAb administration were analyzed by ELISA.

FIGS. 6A-6B show rescue of fentanyl induced antinociception by JBZ-4 and JBZ-7 infusion. Following a 0.2 mg/kg IP dose of fentanyl, mice were tested in hot plate (FIG. 6A) and tail flick (FIG. 6B) antinociception assays at 15 min and immediately administered 30 mg/kg intravenous antibody after which testing resumed. JBZ-4 was superior to JBZ-7.

FIGS. 7A-7B show that JBZ-4 blocks and rescues carfentanil-induced respiratory depression. FIG. 7A: mice pretreated with 30 mg/kg JBZ-4 were challenged 48 h later with IV carfentanil (30 μg/kg) and their respiration was observed by whole body plethysmography and expressed as minute volume (MV) normalized to baseline. FIG. 7B: In rescue experiments, mice were administered IP carfentanil (30 μg/kg) followed by an IV rescue dose of either 1 mg/kg naloxone or (60 mg/kg) JBZ-4 and respiration was observed.

FIGS. 8A-8B are a raw sensorgram of fentanyl (FIG. 8A) and carfentanil (FIG. 8B) association rates (kₒₙ) and dissociation rates (k_{off}) to JBZ-4 with fitted curve.

FIG. 9 shows a concentration-time profile of monoclonal antibody JBZ-4 in rats following a single intravenous bolus dose of JBZ-4 at 10, 100, and 250 mg/kg.

### DETAILED DESCRIPTION

### Definitions

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, N Y 1989). Any methods, devices and materials similar or equivalent to those described herein can be used in the practice of this disclosure. The following definitions are provided to facilitate understanding of certain terms used frequently herein and are not meant to limit the scope of the present disclosure.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“JBZ-1” refers to a monoclonal antibody having the light chain amino acid sequence set forth in SEQ ID NO:45 and the heavy chain amino acid sequence as set forth in SEQ ID NO:46.

“JBZ-2” refers to a monoclonal antibody (mAb) having a light chain variable region as set forth in SEQ ID NO:37 and a heavy chain variable region as set forth in SEQ ID NO:23. In embodiments, the terms refer to a monoclonal antibody (mAb) having a light chain amino acid sequence as set forth in SEQ ID NO:38 and a heavy chain amino acid sequence as set forth in SEQ ID NO:25.

“JBZ-3” refers to the JBZ-2 antibody having six additional mutations in the heavy chain variable region (sequence not shown).

“JBZ-4” refers to an antibody having a light chain variable region comprising CDR L1 as set forth in SEQ ID NO:1, CDR L2 as set forth in SEQ ID NO:2, and CDR L3 as set forth in SEQ ID NO:3; and a heavy chain variable region comprising CDR H1 as set forth in SEQ ID NO:4 or SEQ ID NO:15, CDR H2 as set forth in SEQ ID NO:5 or SEQ ID NO:16, and CDR H3 as set forth in SEQ ID NO:6. In embodiments, the terms refer to an antibody comprising a light chain variable region as set forth in SEQ ID NO:20 and a heavy chain variable region as set forth in SEQ ID NO:23. In embodiments, the terms refer to an antibody comprising a light chain amino acid sequence as set forth in SEQ ID NO:22 and a heavy chain amino acid sequence as set forth in SEQ ID NO:25.

“JBZ-5” refers to an antibody comprising a light chain variable region as set forth in SEQ ID NO:43 and a heavy chain variable region as set forth in SEQ ID NO:23. In embodiments, the term refers to an antibody comprising a light chain amino acid sequence as set forth in SEQ ID NO:44 and a heavy chain amino acid sequence as set forth in SEQ ID NO:25.

“JBZ-6” refers to an antibody comprising a light chain variable region as set forth in SEQ ID NO:40 and a heavy chain variable region as set forth in SEQ ID NO:23. In embodiments, the term refers to an antibody comprising a light chain amino acid sequence as set forth in SEQ ID NO:41 and a heavy chain amino acid sequence as set forth in SEQ ID NO:25.

“JBZ-7” refers to the JBZ-4 antibody having six additional mutations in the heavy chain variable region (sequence not shown).

Six comparative antibodies (sequences not shown) are referred to herein as “P1A2,” “P2C1,” “P2B5,” “P1C1,” “P1D8,” and “P1F8.”

“PBS” refers to phosphate-buffered saline.

Antibodies are large, complex molecules with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen and a constant (“C”) region that interacts with other components of the immune system. The light and heavy chain variable regions come together in 3-dimensional space to form a variable region that binds the antigen (for example, a drug, such as an opioid or a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen (e.g., opioid). The part of a variable region not contained in the CDRs is called the framework (“FR”), which forms the environment for the CDRs. In embodiments, the position of CDRs and FRs are defined herein by the Chothia numbering system (Chothia et al, J. Mol. Biol, 196(4):901-917 (1987); Chothia et al, Nature, 342(6252:877-883 (1989); Al-Lazikani et al, J. Mol. Biol. 273(4):927-948 (1997)). In embodiments, the positions occupied by individual residues within the light or the heavy chain of an antibody are defined herein by the Chothia numbering system. In embodiments, the position of CDRs and FRs are defined herein by the Kabat numbering system (Kabat et al., Sequences of Proteins of Immunological Interest, Fifth Edition, U.S. Department of Health and Human Services, U.S. Government Printing Office (1991)). In embodiments, the positions occupied by individual residues within the light or the heavy chain of an antibody are defined herein by the Kabat numbering system. Throughout this disclosure, the location of residues required for binding within a light chain and a heavy chain of an antibody are defined by the position of the residue according to the Kabat numbering system or the Chothia numbering system, as is well known in the art.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An “antibody variant” as provided herein refers to a polypeptide capable of binding to an antigen and including one or more structural domains of an antibody or fragment thereof. Non-limiting examples of antibody variants include single-domain antibodies or nanobodies, affibodies (polypeptides smaller than monoclonal antibodies (e.g., about 6 kDA) and capable of binding antigens with high affinity and imitating monoclonal antibodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain. Like a whole antibody, it is able to bind selectively to a specific antigen. A “peptibody” as provided herein refers to a peptide moiety attached (through a covalent or non-covalent linker) to the Fc domain of an antibody.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen.

The antibodies described herein, e.g., recombinant, monoclonal, or polyclonal antibodies, may be prepared by any technique known in the art (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. In embodiments, the antibodies described herein are humanized and/or chimeric monoclonal antibodies.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa. In embodiments, the linker includes more than one serine. In embodiments, the linker includes more than one glycine. In embodiments, the linker has the strucutre of —(Gly-Gly-Gly-Gly-Ser)₃-.

The phrase “specifically (or selectively) binds” to an antibody or “specifically (or selectively) immunoreactive with,” when referring to an opioid, protein, or peptide, refers to a binding reaction that is determinative of the presence of the opioid or protein, often in a heterogeneous population of opioids, proteins and other biologics. Thus, under designated immunoassay conditions, the specified antibodies bind to a particular opioid or protein at least two times the background and more typically more than 10 to 100 times background. Specific binding to an antibody under such conditions requires an antibody that is selected for its specificity for a particular opioid or protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular opioid or protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) alanine (A), glycine (G); (2) aspartic acid (D), glutamic acid (E); (3) asparagine (N), glutamine (Q); (4) arginine (R), lysine (K); (5) isoleucine (I), leucine (L), methionine (M), valine (V); (6) phenylalanine (F), tyrosine (Y), tryptophan (W); (7) serine (S), threonine (T); and (8) cysteine (C), methionine (M).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences or individual domains of the polypeptide sequence), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, or less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

An amino acid residue in an antibody “corresponds” to a given residue when it occupies the same essential structural position within the antibody as the given residue. For example, a selected residue in a comparison antibody corresponds to position 48 (according to the Kabat or Chothia numbering system) in an antibody provided herein when the selected residue occupies the same essential spatial or structural relationship to Kabat or Chothia position 48 as assessed using applicable methods in the art. For example, a comparison antibody may be aligned for maximum sequence homology with the antibody provided herein and the position in the aligned comparison antibody that aligns with Kabat or Chothia position 48 may be determined to correspond to it. Alternatively, instead of (or in addition to) a primary sequence alignment as described above, a three dimensional structural alignment can also be used, e.g., where the structure of the comparison antibody is aligned for maximum correspondence with an antibody provided herein and the overall structures compared. In this case, an amino acid that occupies the same essential position as Kabat or Chothia position 48 in the structural model may be said to correspond.

The term “isolated,” when applied to a protein, denotes that the protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a protein gives rise to essentially one band in an electrophoretic gel. Particularly, it means that the protein is at least 85% pure, at least 90% pure, at least 95% pure, or at least 99% pure.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

As used herein, the terms “opioid” and “synthetic opioid” refer to fentanyl, fentanyl analogues, carfentanil, and carfentanil analogues.

“Fentanyl analogue” refers to an analogue of fentanyl. In aspects, a fentanyl analogue is a compound that exhibits mu-opioid receptor binding greater than fentanyl or that exhibits mu-opioid receptor binding in an amount of about 0-50% less than fentanyl, about 0-25% less than fentanyl, or about 0-10% less than fentanyl, based on standard in vitro or in vivo mu-opioid receptor binding assay (e.g., Lipinski et al, Molecules, 24(4):740 (2019)). Exemplary fentanyl analogues include acetylfentanyl, alfentanil, butylfentanyl, butyrfentanyl, para-tolylfentanyl, 3-methylfentanyl, α-methylfentanyl, remifentanil, mefentanyl, phenaridine, ohmefentanyl, mirfentanil, and the like. In aspects, the fentanyl analogue is acetylfentanyl, alfentanil, butylfentanyl, para-tolylfentanyl, 3-methylfentanyl, α-methylfentanyl, or remifentanil. In aspects, the fentanyl analogue is acetylfentanyl. In aspects, the fentanyl analogue is butylfentanyl. In aspects, the fentanyl analogue is alfentanyl. In aspects, the fentanyl analogue is remifentanil. In aspects, the fentanyl analogue is butyrfentanyl. In aspects, the fentanyl analogue is para-tolylfentanyl. In aspects, the fentanyl analogue is 3-methylfentanyl. In aspects, the fentanyl analogue is α-methylfentanyl. In aspects, the fentanyl analogue is mefentanyl. In aspects, the fentanyl analogue is phenaridine. In aspects, the fentanyl analogue is ohmefentanyl. In aspects, the fentanyl analogue is mirfentanil.

“Carfentanil analogue” refers to an analogue of carfentanil. In aspects, a carfentanil analogue is a compound that exhibits mu-opioid receptor binding greater than carfentanil or that exhibits mu-opioid receptor binding in an amount of about 0-50% less than carfentanil, about 0-25% less than carfentanil, or about 0-10% less than carfentanil, based on a standard in vitro or in vivo mu-opioid receptor binding assay (e.g., Lipinski et al, Molecules, 24(4):740 (2019)). Exemplary carfentanil analogues include sufentanil, remifentanil, alfentanil, lofentanil, brifentanil, trefentanil, and the like. In aspects, the carfentanil analogue is sufentanil. In aspects, the carfentanil analogue is remifentanil. In aspects, the carfentanil analogue is alfentanil. In aspects, the carfentanil analogue is lofentanil. In aspects, the carfentanil analogue is brifentanil. In aspects, the carfentanil analogue is trefentanil.

Antibodies

Provided herein is an antibody comprising: (i) a light chain variable region which comprises a CDR L1 having an amino acid sequence with at least 85%, 88%, 90%, 91%, 92%, 93%, 94%, 95%, 96%, 97%, 98%, or 99% sequence identity to SEQ ID NO:1, a CDR L2 having an amino acid sequence with at least 85%, 88%, 90%, 91%, 92%, 93%, 94%, 95%, 96%, 97%, 98%, or 99% sequence identity to SEQ ID NO:2, and a CDR L3 having an amino acid sequence with at least 85%, 88%, 90%, 91%, 92%, 93%, 94%, 95%, 96%, 97%, 98%, or 99% sequence identity to SEQ ID NO:3; and (ii) a heavy chain variable region which comprises a CDR H1 having an amino acid sequence with at least 85%, 88%, 90%, 91%, 92%, 93%, 94%, 95%, 96%, 97%, 98%, or 99% sequence identity to SEQ ID NO:4, a CDR H2 having an amino acid sequence with at least 85%, 88%, 90%, 91%, 92%, 93%, 94%, 95%, 96%, 97%, 98%, or 99% sequence identity to SEQ ID NO:5, and a CDR H3 having an amino acid sequence with at least 85%, 88%, 90%, 91%, 92%, 93%, 94%, 95%, 96%, 97%, 98%, or 99% sequence identity to SEQ ID NO:6. In embodiments, the antibody further comprises a light chain framework region which comprises a LFR1 having an amino acid sequence with at least 85%, 88%, 90%, 91%, 92%, 93%, 94%, 95%, 96%, 97%, 98%, or 99% sequence identity to SEQ ID NO:7, a LFR2 having an amino acid sequence with at least 85%, 88%, 90%, 91%, 92%, 93%, 94%, 95%, 96%, 97%, 98%, or 99% sequence identity to SEQ ID NO:8, a LFR3 having an amino acid sequence with at least 85%, 88%, 90%, 91%, 92%, 93%, 94%, 95%, 96%, 97%, 98%, or 99% sequence identity to SEQ ID NO:9, and a LFR4 having an amino acid sequence with at least 85%, 88%, 90%, 91%, 92%, 93%, 94%, 95%, 96%, 97%, 98%, or 99% sequence identity to SEQ ID NO:10. In embodiments, the antibody further comprises a heavy chain framework region which comprises a HFR1 having an amino acid sequence with at least 85%, 88%, 90%, 91%, 92%, 93%, 94%, 95%, 96%, 97%, 98%, or 99% sequence identity to SEQ ID NO:11; a HFR2 having an amino acid sequence with at least 85%, 88%, 90%, 91%, 92%, 93%, 94%, 95%, 96%, 97%, 98%, or 99% sequence identity to SEQ ID NO:12, a HFR3 having an amino acid sequence with at least 85%, 88%, 90%, 91%, 92%, 93%, 94%, 95%, 96%, 97%, 98%, or 99% sequence identity to SEQ ID NO:13, and a HFR4 having an amino acid sequence with at least 85%, 88%, 90%, 91%, 92%, 93%, 94%, 95%, 96%, 97%, 98%, or 99% sequence identity to SEQ ID NO:14. In embodiments, SEQ ID NOS:1-14 are based on the Kabat numbering system. In embodiments, the antibody has a binding affinity to fentanyl that is greater than or equal to the binding affinity to fentanyl of the antibody having the light chain sequence of SEQ ID NO:22 and the heavy chain sequence of SEQ ID NO:25. In embodiments, the antibody has a binding affinity to fentanyl that is 0% to 25% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 20% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 15% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 10% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 5% less than the binding affinity to fentanyl of the antibody having the light chain sequence of SEQ ID NO:22 and the heavy chain sequence of SEQ ID NO:25. In embodiments, the antibody is an IgG antibody. In embodiments, the antibody is an IgG1 antibody. In embodiments, the antibody is a Fab′ fragment. In embodiments, the antibody is a F(ab)′2 fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising: (i) a light chain variable region which comprises a CDR L1 having an amino acid sequence with at least 90% sequence identity to SEQ ID NO:1, a CDR L2 having an amino acid sequence with at least 90% sequence identity to SEQ ID NO:2, and a CDR L3 having an amino acid sequence with at least 90% sequence identity to SEQ ID NO:3; and (ii) a heavy chain variable region which comprises a CDR H1 having an amino acid sequence with at least 90% sequence identity to SEQ ID NO:4, a CDR H2 having an amino acid sequence with at least 90% sequence identity to SEQ ID NO:5, and a CDR H3 having an amino acid sequence with at least 90% sequence identity to SEQ ID NO:6. In embodiments, the antibody comprises: (i) a light chain variable region which comprises a CDR L1 having an amino acid sequence with at least 92% sequence identity to SEQ ID NO:1, a CDR L2 having an amino acid sequence with at least 92% sequence identity to SEQ ID NO:2, and a CDR L3 having an amino acid sequence with at least 92% sequence identity to SEQ ID NO:3; and (ii) a heavy chain variable region which comprises a CDR H1 having an amino acid sequence with at least 92% sequence identity to SEQ ID NO:4, a CDR H2 having an amino acid sequence with at least 92% sequence identity to SEQ ID NO:5, and a CDR H3 having an amino acid sequence with at least 92% sequence identity to SEQ ID NO:6. In embodiments, the antibody comprises: (i) a light chain variable region which comprises a CDR L1 having an amino acid sequence with at least 94% sequence identity to SEQ ID NO:1, a CDR L2 having an amino acid sequence with at least 94% sequence identity to SEQ ID NO:2, and a CDR L3 having an amino acid sequence with at least 94% sequence identity to SEQ ID NO:3; and (ii) a heavy chain variable region which comprises a CDR H1 having an amino acid sequence with at least 94% sequence identity to SEQ ID NO:4, a CDR H2 having an amino acid sequence with at least 94% sequence identity to SEQ ID NO:5, and a CDR H3 having an amino acid sequence with at least 94% sequence identity to SEQ ID NO:6. In embodiments, the antibody comprises: (i) a light chain variable region which comprises a CDR L1 having an amino acid sequence with at least 95% sequence identity to SEQ ID NO:1, a CDR L2 having an amino acid sequence with at least 95% sequence identity to SEQ ID NO:2, and a CDR L3 having an amino acid sequence with at least 95% sequence identity to SEQ ID NO:3; and (ii) a heavy chain variable region which comprises a CDR H1 having an amino acid sequence with at least 95% sequence identity to SEQ ID NO:4, a CDR H2 having an amino acid sequence with at least 95% sequence identity to SEQ ID NO:5, and a CDR H3 having an amino acid sequence with at least 95% sequence identity to SEQ ID NO:6. In embodiments, the antibody comprises: (i) a light chain variable region which comprises a CDR L1 having an amino acid sequence with at least 96% sequence identity to SEQ ID NO:1, a CDR L2 having an amino acid sequence with at least 96% sequence identity to SEQ ID NO:2, and a CDR L3 having an amino acid sequence with at least 96% sequence identity to SEQ ID NO:3; and (ii) a heavy chain variable region which comprises a CDR H1 having an amino acid sequence with at least 96% sequence identity to SEQ ID NO:4, a CDR H2 having an amino acid sequence with at least 96% sequence identity to SEQ ID NO:5, and a CDR H3 having an amino acid sequence with at least 96% sequence identity to SEQ ID NO:6. In embodiments, the antibody comprises: (i) a light chain variable region which comprises a CDR L1 having an amino acid sequence with at least 98% sequence identity to SEQ ID NO:1, a CDR L2 having an amino acid sequence with at least 98% sequence identity to SEQ ID NO:2, and a CDR L3 having an amino acid sequence with at least 98% sequence identity to SEQ ID NO:3; and (ii) a heavy chain variable region which comprises a CDR H1 having an amino acid sequence with at least 98% sequence identity to SEQ ID NO:4, a CDR H2 having an amino acid sequence with at least 98% sequence identity to SEQ ID NO:5, and a CDR H3 having an amino acid sequence with at least 98% sequence identity to SEQ ID NO:6. In embodiments, the antibody is an IgG antibody. In embodiments, the antibody is an IgG1 antibody. In embodiments, the antibody is a Fab′ fragment. In embodiments, the antibody is a F(ab)′2 fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

In embodiments, the antibody further comprises a light chain framework region which comprises a LFR1 having an amino acid sequence with at least 90% sequence identity to SEQ ID NO:7, a LFR2 having an amino acid sequence with at least 90% sequence identity to SEQ ID NO:8, a LFR3 having an amino acid sequence with at least 90% sequence identity to SEQ ID NO:9, and a LFR4 having an amino acid sequence with at least 90% sequence identity to SEQ ID NO:10. In embodiments, the antibody further comprises a heavy chain framework region which comprises a HFR1 having an amino acid sequence with at least 90% sequence identity to SEQ ID NO:11; a HFR2 having an amino acid sequence with at least 90% sequence identity to SEQ ID NO:12, a HFR3 having an amino acid sequence with at least 90% sequence identity to SEQ ID NO:13, and a HFR4 having an amino acid sequence with at least 90% sequence identity to SEQ ID NO:14. In embodiments, the antibody comprises: (i) a light chain variable region which comprises a CDR L1 having an amino acid sequence with at least 95% sequence identity to SEQ ID NO:1, a CDR L2 having an amino acid sequence with at least 95% sequence identity to SEQ ID NO:2, and a CDR L3 having an amino acid sequence with at least 95% sequence identity to SEQ ID NO:3; and (ii) a heavy chain variable region which comprises a CDR H1 having an amino acid sequence with at least 95% sequence identity to SEQ ID NO:4, a CDR H2 having an amino acid sequence with at least 95% sequence identity to SEQ ID NO:5, and a CDR H3 having an amino acid sequence with at least 95% sequence identity to SEQ ID NO:6. In embodiments, the antibody further comprises a light chain framework region which comprises a LFR1 having an amino acid sequence with at least 95% sequence identity to SEQ ID NO:7, a LFR2 having an amino acid sequence with at least 95% sequence identity to SEQ ID NO:8, a LFR3 having an amino acid sequence with at least 95% sequence identity to SEQ ID NO:9, and a LFR4 having an amino acid sequence with at least 95% sequence identity to SEQ ID NO:10. In embodiments, the antibody further comprises a heavy chain framework region which comprises a HFR1 having an amino acid sequence with at least 95% sequence identity to SEQ ID NO:11; a HFR2 having an amino acid sequence with at least 95% sequence identity to SEQ ID NO:12, a HFR3 having an amino acid sequence with at least 95% sequence identity to SEQ ID NO:13, and a HFR4 having an amino acid sequence with at least 95% sequence identity to SEQ ID NO:14. In embodiments, the antibody is an IgG antibody. In embodiments, the antibody is an IgG1 antibody. In embodiments, the antibody is a Fab′ fragment. In embodiments, the antibody is a F(ab)′2 fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising: (i) a light chain variable region which comprises a CDR L1 as set forth in SEQ ID NO:1 and having one or more conservatively modified variants; a CDR L2 as set forth in SEQ ID NO:2 and having one or more conservatively modified variants; and a CDR L3 as set forth in SEQ ID NO:3 and having one or more conservatively modified variants; and (ii) a heavy chain variable region which comprises a CDR H1 as set forth in SEQ ID NO:4 and having one or more conservatively modified variants; a CDR H2 as set forth in SEQ ID NO:5 and having one or more conservatively modified variants; and a CDR H3 as set forth in SEQ ID NO:6 and having one or more conservatively modified variants. In embodiments, the antibody further comprises a light chain framework region which comprises a LFR1 as set forth in SEQ ID NO:7 and having one or more conservatively modified variants; a LFR2 as set forth in SEQ ID NO:8 and having one or more conservatively modified variants; a LFR3 as set forth in SEQ ID NO:9 and having one or more conservatively modified variants; and a LFR4 as set forth in SEQ ID NO:10 and having one or more conservatively modified variants. In embodiments, the antibody further comprises a heavy chain framework region which comprises a HFR1 as set forth in SEQ ID NO:11 and having one or more conservatively modified variants; a HFR2 as set forth in SEQ ID NO:12 and having one or more conservatively modified variants; a HFR3 as set forth in SEQ ID NO:13 and having one or more conservatively modified variants; and a HFR4 as set forth in SEQ ID NO:14 and having one or more conservatively modified variants. In embodiments, one or more conservatively modified variants is independently selected from 1 conservatively modified variant to about 10 conservatively modified variants. In embodiments, one or more conservatively modified variants is independently selected from 1 conservatively modified variant to about 9 conservatively modified variants. In embodiments, one or more conservatively modified variants is independently selected from 1 conservatively modified variant to about 8 conservatively modified variants. In embodiments, one or more conservatively modified variants is independently selected from 1 conservatively modified variant to about 7 conservatively modified variants. In embodiments, one or more conservatively modified variants is independently selected from 1 conservatively modified variant to about 6 conservatively modified variants. In embodiments, one or more conservatively modified variants is independently selected from 1 conservatively modified variant to about 5 conservatively modified variants. In embodiments, one or more conservatively modified variants is independently selected from 1 conservatively modified variant to about 4 conservatively modified variants. In embodiments, one or more conservatively modified variants is independently selected from 1 conservatively modified variant to about 3 conservatively modified variants. In embodiments, one or more conservatively modified variants is independently selected from 1 or 2 conservatively modified variant. In embodiments, one or more conservatively modified variants is 1 conservatively modified variant. In embodiments, one or more conservatively modified variants is 2 conservatively modified variants. In embodiments, one or more conservatively modified variants is 3 conservatively modified variants. In embodiments, one or more conservatively modified variants is 4 conservatively modified variants. In embodiments, one or more conservatively modified variants is 5 conservatively modified variants. As described herein, a conservatively modified variant includes a conservative substitution, deletion of an amino acid, addition of an amino acid, or a combination of two or more thereof. In embodiments, a conservatively modified variation is a conservative substitution. In embodiments, a conservatively modified variation is a deletion of an amino acid. In embodiments, a conservatively modified variation is an addition of an amino acid. In embodiments, the antibody has a binding affinity to fentanyl that is greater than or equal to the binding affinity to fentanyl of the antibody having the light chain sequence of SEQ ID NO:22 and the heavy chain sequence of SEQ ID NO:25. In embodiments, the antibody has a binding affinity to fentanyl that is 0% to 25% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 20% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 15% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 10% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 5% less than the binding affinity to fentanyl of the antibody having the light chain sequence of SEQ ID NO:22 and the heavy chain sequence of SEQ ID NO:25. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising: (i) a light chain variable region which comprises a CDR L1 as set forth in SEQ ID NO:1, a CDR L2 as set forth in SEQ ID NO:2, and a CDR L3 as set forth in SEQ ID NO:3; and (ii) a heavy chain variable region which comprises a CDR H1 as set forth in SEQ ID NO:4, a CDR H2 as set forth in SEQ ID NO:5, and a CDR H3 as set forth in SEQ ID NO:6. In embodiments, the antibody further comprises a light chain framework region which comprises a LFR1 as set forth in SEQ ID NO:7, a LFR2 as set forth in SEQ ID NO:8, a LFR3 as set forth in SEQ ID NO:9, and a LFR4 as set forth in SEQ ID NO:10. In embodiments, the antibody further comprises a heavy chain framework region which comprises a HFR1 as set forth in SEQ ID NO:11; a HFR2 as set forth in SEQ ID NO:12, a HFR3 as set forth in SEQ ID NO:13, and a HFR4 as set forth in SEQ ID NO:14. In embodiments, the antibody is an IgG antibody. In embodiments, the antibody is an IgG1 antibody. In embodiments, the antibody is a Fab′ fragment. In embodiments, the antibody is a F(ab)′2 fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising: (i) a light chain variable region which comprises a CDR L1 having an amino acid sequence with at least 85%, 88%, 90%, 91%, 92%, 93%, 94%, 95%, 96%, 97%, 98%, or 99% sequence identity to SEQ ID NO:1, a CDR L2 having an amino acid sequence with at least 85%, 88%, 90%, 91%, 92%, 93%, 94%, 95%, 96%, 97%, 98%, or 99% sequence identity to SEQ ID NO:2, and a CDR L3 having an amino acid sequence with at least 85%, 88%, 90%, 91%, 92%, 93%, 94%, 95%, 96%, 97%, 98%, or 99% sequence identity to SEQ ID NO:3; and (ii) a heavy chain variable region which comprises a CDR H1 having an amino acid sequence with at least 85%, 88%, 90%, 91%, 92%, 93%, 94%, 95%, 96%, 97%, 98%, or 99% sequence identity to SEQ ID NO:15; a CDR H2 having an amino acid sequence with at least 85%, 88%, 90%, 91%, 92%, 93%, 94%, 95%, 96%, 97%, 98%, or 99% sequence identity to SEQ ID NO:16, and a CDR H3 having an amino acid sequence with at least 85%, 88%, 90%, 91%, 92%, 93%, 94%, 95%, 96%, 97%, 98%, or 99% sequence identity to SEQ ID NO:6. In embodiments, the antibody further comprises a light chain framework region which comprises a LFR1 having an amino acid sequence with at least 85%, 88%, 90%, 91%, 92%, 93%, 94%, 95%, 96%, 97%, 98%, or 99% sequence identity to SEQ ID NO:7, a LFR2 having an amino acid sequence with at least 85%, 88%, 90%, 91%, 92%, 93%, 94%, 95%, 96%, 97%, 98%, or 99% sequence identity to SEQ ID NO:8, a LFR3 having an amino acid sequence with at least 85%, 88%, 90%, 91%, 92%, 93%, 94%, 95%, 96%, 97%, 98%, or 99% sequence identity to SEQ ID NO:9, and a LFR4 having an amino acid sequence with at least 85%, 88%, 90%, 91%, 92%, 93%, 94%, 95%, 96%, 97%, 98%, or 99% sequence identity to SEQ ID NO:10. In embodiments, the antibody further comprises a heavy chain framework region which comprises a HFR1 having an amino acid sequence with at least 85%, 88%, 90%, 91%, 92%, 93%, 94%, 95%, 96%, 97%, 98%, or 99% sequence identity to SEQ ID NO:17; a HFR2 having an amino acid sequence with at least 85%, 88%, 90%, 91%, 92%, 93%, 94%, 95%, 96%, 97%, 98%, or 99% sequence identity to SEQ ID NO:18, a HFR3 having an amino acid sequence with at least 85%, 88%, 90%, 91%, 92%, 93%, 94%, 95%, 96%, 97%, 98%, or 99% sequence identity to SEQ ID NO:19, and a HFR4 having an amino acid sequence with at least 85%, 88%, 90%, 91%, 92%, 93%, 94%, 95%, 96%, 97%, 98%, or 99% sequence identity to SEQ ID NO:14. In embodiments, SEQ ID NOS:1-3, 6-10, 14-16, and 17-19 are based on the Chothia numbering system. In embodiments, the antibody has a binding affinity to fentanyl that is greater than or equal to the binding affinity to fentanyl of the antibody having the light chain sequence of SEQ ID NO:22 and the heavy chain sequence of SEQ ID NO:25. In embodiments, the antibody has a binding affinity to fentanyl that is 0% to 25% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 20% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 15% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 10% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 5% less than the binding affinity to fentanyl of the antibody having the light chain sequence of SEQ ID NO:22 and the heavy chain sequence of SEQ ID NO:25. In embodiments, the binding affinity is determined by the assay set forth in Example 3. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising: (i) a light chain variable region which comprises a CDR L1 having an amino acid sequence with at least 90% sequence identity to SEQ ID NO:1, a CDR L2 having an amino acid sequence with at least 90% sequence identity to SEQ ID NO:2, and a CDR L3 having an amino acid sequence with at least 90% sequence identity to SEQ ID NO:3; and (ii) a heavy chain variable region which comprises a CDR H1 having an amino acid sequence with at least 90% sequence identity to SEQ ID NO:15, a CDR H2 having an amino acid sequence with at least 90% sequence identity to SEQ ID NO:16, and a CDR H3 having an amino acid sequence with at least 90% sequence identity to SEQ ID NO:6. In embodiments, the antibody comprises (i) a light chain variable region which comprises a CDR L1 having an amino acid sequence with at least 92% sequence identity to SEQ ID NO:1, a CDR L2 having an amino acid sequence with at least 92% sequence identity to SEQ ID NO:2, and a CDR L3 having an amino acid sequence with at least 92% sequence identity to SEQ ID NO:3; and (ii) a heavy chain variable region which comprises a CDR H1 having an amino acid sequence with at least 92% sequence identity to SEQ ID NO:15, a CDR H2 having an amino acid sequence with at least 92% sequence identity to SEQ ID NO:16, and a CDR H3 having an amino acid sequence with at least 92% sequence identity to SEQ ID NO:6. In embodiments, the antibody comprises (i) a light chain variable region which comprises a CDR L1 having an amino acid sequence with at least 94% sequence identity to SEQ ID NO:1, a CDR L2 having an amino acid sequence with at least 94% sequence identity to SEQ ID NO:2, and a CDR L3 having an amino acid sequence with at least 94% sequence identity to SEQ ID NO:3; and (ii) a heavy chain variable region which comprises a CDR H1 having an amino acid sequence with at least 94% sequence identity to SEQ ID NO:15, a CDR H2 having an amino acid sequence with at least 94% sequence identity to SEQ ID NO:16, and a CDR H3 having an amino acid sequence with at least 94% sequence identity to SEQ ID NO:6. In embodiments, the antibody comprises: (i) a light chain variable region which comprises a CDR L1 having an amino acid sequence with at least 95% sequence identity to SEQ ID NO:1, a CDR L2 having an amino acid sequence with at least 95% sequence identity to SEQ ID NO:2, and a CDR L3 having an amino acid sequence with at least 95% sequence identity to SEQ ID NO:3; and (ii) a heavy chain variable region which comprises a CDR H1 having an amino acid sequence with at least 95% sequence identity to SEQ ID NO:15, a CDR H2 having an amino acid sequence with at least 95% sequence identity to SEQ ID NO:16, and a CDR H3 having an amino acid sequence with at least 95% sequence identity to SEQ ID NO:6. In embodiments, the antibody comprises (i) a light chain variable region which comprises a CDR L1 having an amino acid sequence with at least 96% sequence identity to SEQ ID NO:1, a CDR L2 having an amino acid sequence with at least 96% sequence identity to SEQ ID NO:2, and a CDR L3 having an amino acid sequence with at least 96% sequence identity to SEQ ID NO:3; and (ii) a heavy chain variable region which comprises a CDR H1 having an amino acid sequence with at least 96% sequence identity to SEQ ID NO:15, a CDR H2 having an amino acid sequence with at least 96% sequence identity to SEQ ID NO:16, and a CDR H3 having an amino acid sequence with at least 96% sequence identity to SEQ ID NO:6. In embodiments, the antibody comprises (i) a light chain variable region which comprises a CDR L1 having an amino acid sequence with at least 98% sequence identity to SEQ ID NO:1, a CDR L2 having an amino acid sequence with at least 98% sequence identity to SEQ ID NO:2, and a CDR L3 having an amino acid sequence with at least 98% sequence identity to SEQ ID NO:3; and (ii) a heavy chain variable region which comprises a CDR H1 having an amino acid sequence with at least 98% sequence identity to SEQ ID NO:15, a CDR H2 having an amino acid sequence with at least 98% sequence identity to SEQ ID NO:16, and a CDR H3 having an amino acid sequence with at least 98% sequence identity to SEQ ID NO:6. In embodiments, the antibody is an IgG antibody. In embodiments, the antibody is an IgG1 antibody. In embodiments, the antibody is a Fab′ fragment. In embodiments, the antibody is a F(ab)′2 fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

In embodiments, the antibody further comprises a light chain framework region which comprises a LFR1 having an amino acid sequence with at least 90% sequence identity to SEQ ID NO:7, a LFR2 having an amino acid sequence with at least 90% sequence identity to SEQ ID NO:8, a LFR3 having an amino acid sequence with at least 90% sequence identity to SEQ ID NO:9, and a LFR4 having an amino acid sequence with at least 90% sequence identity to SEQ ID NO:10. In embodiments, the antibody further comprises a heavy chain framework region which comprises a HFR1 having an amino acid sequence with at least 90% sequence identity to SEQ ID NO:17; a HFR2 having an amino acid sequence with at least 90% sequence identity to SEQ ID NO:18, a HFR3 having an amino acid sequence with at least 90% sequence identity to SEQ ID NO:19, and a HFR4 having an amino acid sequence with at least 90% sequence identity to SEQ ID NO:14. In embodiments, the antibody further comprises a light chain framework region which comprises a LFR1 having an amino acid sequence with at least 95% sequence identity to SEQ ID NO:7, a LFR2 having an amino acid sequence with at least 95% sequence identity to SEQ ID NO:8, a LFR3 having an amino acid sequence with at least 95% sequence identity to SEQ ID NO:9, and a LFR4 having an amino acid sequence with at least 95% sequence identity to SEQ ID NO:10. In embodiments, the antibody further comprises a heavy chain framework region which comprises a HFR1 having an amino acid sequence with at least 95% sequence identity to SEQ ID NO:17; a HFR2 having an amino acid sequence with at least 95% sequence identity to SEQ ID NO:18, a HFR3 having an amino acid sequence with at least 95% sequence identity to SEQ ID NO:19, and a HFR4 having an amino acid sequence with at least 95% sequence identity to SEQ ID NO:14. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising: (i) a light chain variable region which comprises a CDR L1 as set forth in SEQ ID NO:1 and having one or more conservatively modified variants; a CDR L2 as set forth in SEQ ID NO:2 and having one or more conservatively modified variants; and a CDR L3 as set forth in SEQ ID NO:3 and having one or more conservatively modified variants; and (ii) a heavy chain variable region which comprises a CDR H1 as set forth in SEQ ID NO:15 and having one or more conservatively modified variants; a CDR H2 as set forth in SEQ ID NO:16 and having one or more conservatively modified variants; and a CDR H3 as set forth in SEQ ID NO:6 and having one or more conservatively modified variants. In embodiments, the antibody further comprises a light chain framework region which comprises a LFR1 as set forth in SEQ ID NO:7 and having one or more conservatively modified variants; a LFR2 as set forth in SEQ ID NO:8 and having one or more conservatively modified variants; a LFR3 as set forth in SEQ ID NO:9 and having one or more conservatively modified variants; and a LFR4 as set forth in SEQ ID NO:10 and having one or more conservatively modified variants. In embodiments, the antibody further comprises a heavy chain framework region which comprises a HFR1 as set forth in SEQ ID NO:17 and having one or more conservatively modified variants; a HFR2 as set forth in SEQ ID NO:18 and having one or more conservatively modified variants; a HFR3 as set forth in SEQ ID NO:19 and having one or more conservatively modified variants; and a HFR4 as set forth in SEQ ID NO:14 and having one or more conservatively modified variants. In embodiments, one or more conservatively modified variants is independently selected from 1 conservatively modified variant to about 5 conservatively modified variants. In embodiments, one or more conservatively modified variants is independently selected from 1 conservatively modified variant to about 4 conservatively modified variants. In embodiments, one or more conservatively modified variants is independently selected from 1 conservatively modified variant to about 3 conservatively modified variants. In embodiments, one or more conservatively modified variants is independently selected from 1 or 2 conservatively modified variant. In embodiments, one or more conservatively modified variants is 1 conservatively modified variant. In embodiments, one or more conservatively modified variants is 2 conservatively modified variants. In embodiments, one or more conservatively modified variants is 3 conservatively modified variants. In embodiments, one or more conservatively modified variants is 4 conservatively modified variants. In embodiments, one or more conservatively modified variants is 5 conservatively modified variants. As described herein, a conservatively modified variant includes a conservative substitution, deletion of an amino acid, addition of an amino acid, or a combination of two or more thereof. In embodiments, a conservatively modified variation is a conservative substitution. In embodiments, a conservatively modified variation is a deletion of an amino acid. In embodiments, a conservatively modified variation is an addition of an amino acid. In embodiments, the antibody has a binding affinity to fentanyl that is greater than or equal to the binding affinity to fentanyl of the antibody having the light chain sequence of SEQ ID NO:22 and the heavy chain sequence of SEQ ID NO:25. In embodiments, the antibody has a binding affinity to fentanyl that is 0% to 25% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 20% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 15% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 10% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 5% less than the binding affinity to fentanyl of the antibody having the light chain sequence of SEQ ID NO:22 and the heavy chain sequence of SEQ ID NO:25. In embodiments, the binding affinity is determined by the assay set forth in Example 3. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising: (i) a light chain variable region which comprises a CDR L1 as set forth in SEQ ID NO:1, a CDR L2 as set forth in SEQ ID NO:2, and a CDR L3 as set forth in SEQ ID NO:3; and (ii) a heavy chain variable region which comprises a CDR H1 as set forth in SEQ ID NO:15, a CDR H2 as set forth in SEQ ID NO:16, and a CDR H3 as set forth in SEQ ID NO:6. In embodiments, the antibody further comprises a light chain framework region which comprises a LFR1 as set forth in SEQ ID NO:7, a LFR2 as set forth in SEQ ID NO: 8, a LFR3 as set forth in SEQ ID NO:9, and a LFR4 as set forth in SEQ ID NO:10. In embodiments, the antibody further comprises a heavy chain framework region which comprises a HFR1 as set forth in SEQ ID NO:17; a HFR2 as set forth in SEQ ID NO:18, a HFR3 as set forth in SEQ ID NO:19, and a HFR4 as set forth in SEQ ID NO:14. I In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising: (i) a light chain variable region which comprises a CDR L1 having the sequence xSxGxDxTxLxPxKxRxSxGxYx, a CDR L2 having the sequence xKxDxTxExRxPxSx, and a CDR L3 having the sequence xQxSxAxDxSxSxFxTxYxPxSx; and (ii) a heavy chain variable region which comprises a CDR H1 having the sequence xSxRxNxWxWxSx or xGxGxFxIxSxSxRxNx, a CDR H2 having the sequence xExVxYxHxTxGxIxTxNxYxNxPxSxLxKxSx or xYxHxTxGxlx, and a CDR H3 having the sequence xExVxVxGxPxTxTxGxYxFxDxLx; wherein “x” is an amino acid or absent. In embodiments, the antibody further comprises a light chain framework region which comprises a LFR1 having the sequence xSxYxExLxTxQxPxPxSxVxSxVxSxPxGxQxTxAxRxIxTxCx, a LFR2 having the sequence xWxYxQxQxKxPxDxQxAxPxLxLxVxIxNx, a LFR3 having the sequence xGxIxPxExRxFxSxGxSxKxSxGxTxTxVxTxLxTxIxSxGxVxQxAxExDxExAxDxYxYxCx, and a LFR4 having the sequence xFxGxGxGxTxKxLxTxVxLx; wherein “x” is an amino acid or absent. In embodiments, the antibody further comprises a heavy chain framework region which comprises a HFR1 having the sequence xQxVxQxLxQxExSxGxPxGxLxVxKxPxSxGxTxLxSxLxTxCxTxVxSxGxGxFxIxSx or xQxVxQxLxQxExSxGxPxGxLxVxKxPxSxGxTxLxSxLxTxCxTxVxSx; a HFR2 having the sequence xWxVxRxQxPxPxGxKxGxLxExWxIxGx or xWxWxSxWxVxRxQxPxPxGxKxGxLxExWxIxGxExVx, a HFR3 having the sequence xRxVxTxIxSxVxDxKxSxKxNxQxFxSxLxKxLxSxSxVxTxAxAxDxTxAxVxYxYxCxAxRx or xTxNxYxNxPxSxLxKxSxRxVxTxIxSxVxDxKxSxKxNxQxFxSxLxKxLxSxSxVxTxAxAxDx TxAxVxYxYxCxAxRx, and a HFR4 having the sequence xWxGxRxGxTxLxVxTxIxSxSx; wherein “x” is an amino acid or absent. In embodiments, the antibody has a binding affinity to fentanyl that is greater than or equal to the binding affinity to fentanyl of the antibody having the light chain sequence of SEQ ID NO:22 and the heavy chain sequence of SEQ ID NO:25. In embodiments, the antibody has a binding affinity to fentanyl that is 0% to 25% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 20% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 15% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 10% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 5% less than the binding affinity to fentanyl of the antibody having the light chain sequence of SEQ ID NO:22 and the heavy chain sequence of SEQ ID NO:25. In embodiments, the binding affinity is determined by the assay set forth in Example 3. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising a light chain variable region which comprises: (i) a CDR L1 as set forth in SEQ ID NO:1; (ii) a CDR L2 as set forth in SEQ ID NO:2; and (iii) a CDR L3 as set forth in SEQ ID NO:3, a CDR L3 having the sequence xQSAxFTYPx; or a CDR L3 having the sequence xQSADSSFTYPSx; and a heavy chain variable region which comprises: (iv) a CDR H1 as set forth in SEQ ID NO:4, a CDR H1 as set forth in SEQ ID NO:15, or a CDR H1 having the sequence GGFISSRNW; (v) a CDR H2 as set forth in SEQ ID NO:5, a CDR H2 as set forth in SEQ ID NO: 16, a CDR H2 having the sequence xWxExIxNx, or a CDR H2 having the sequence xWxExITNx; and (vi) a CDR H3 as set forth in SEQ ID NO:6, a CDR H3 having the sequence xExVxGYFx, or a CDR H3 having the sequence xExVGPxTGYFx; wherein x is independently absent or 1 to 8 amino acids. In embodiments, CDR L3 has the amino acid sequence of SEQ ID NO:3. In embodiments, CDR L3 has the sequence xQSAxFTYPx. In embodiments, CDR L3 has the sequence xQSADSSFTYPSx. In embodiments, CDR H1 is as set forth in SEQ ID NO:4. In embodiments, CDR H1 has the sequence GGFISSRNW. In embodiments, CDR H1 is as set forth in SEQ ID NO:15. In embodiments, CDR H2 is as set forth in SEQ ID NO:5. In embodiments, CDR H2 is as set forth in SEQ ID NO:16. In embodiments, CDR H2 has the sequence xWxExIxNx. In embodiments, CDR H2 has the sequence xWxExITNx. In embodiments, CDR H3 is as set forth in SEQ ID NO:6. In embodiments, CDR H3 has the sequence xExVxGYFx. In embodiments, CDR H3 has the sequence ExVGPxTGYFx. In embodiments, x is independently absent or 1 to 7 amino acids. In embodiments, x is independently absent or 1 to 6 amino acids. In embodiments, x is independently absent or 1 to 5 amino acids. In embodiments, x is independently absent or 1 to 4 amino acids. In embodiments, x is independently absent or 1 to 3 amino acids. In embodiments, x is independently absent or 1 to 2 amino acids. In embodiments, x is independently absent or 1 amino acid. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

Provided herein is an antibody comprising a light chain variable region which comprises (i) a CDR L1 as set forth in SEQ ID NO:1; (ii) a CDR L2 as set forth in SEQ ID NO:2; and (iii) a CDR L3 as set forth in SEQ ID NO:3, a CDR L3 having the sequence QSADSSFTYP; a CDR L3 having the sequence QSA; a CDR L3 having the sequence QSAD; a CDR L3 having the sequence QSADS; a CDR L3 having the sequence QSADSS; a CDR L3 having the sequence FTYP; a CDR L3 having the sequence SFTYP; a CDR L3 having the sequence SSFTYP; or a CDR L3 having the sequence DSSFTYP; and a heavy chain variable region which comprises (iv) a CDR H1 as set forth in SEQ ID NO:4, a CDR H1 as set forth in SEQ ID NO:15, or a CDR H1 having the sequence GGFISSRNW; (v) a CDR H2 as set forth in SEQ ID NO:5, a CDR H2 as set forth in SEQ ID NO: 16, a CDR H2 having the sequence WIGEVYHTGI, a CDR H2 having the sequence EVYHTGI, a CDR H2 having the sequence WIGEVYHTGITN, a CDR H2 having the sequence WIGEVYHTGIT, a CDR H2 having the sequence EVYHTGITN, a CDR H2 having the sequence YHTGITN, a CDR H2 having the sequence ITN; and (vi) a CDR H3 as set forth in SEQ ID NO:6, a CDR H3 having the sequence EVV, a CDR H3 having the sequence EVVG, a CDR H3 having the sequence EVVGP, a CDR H3 having the sequence EVVGPT, a CDR H3 having the sequence EVVGPTT, a CDR H3 having the sequence EVVGPTTG, a CDR H3 having the sequence EVVGPTTGY, a CDR H3 having the sequence VGP, a CDR H3 having the sequence GYF, a CDR H3 having the sequence TGYF, a CDR H3 having the sequence TTGYF, a CDR H3 having the sequence PTTGYF, a CDR H3 having the sequence GPTTGYF, a CDR H3 having the sequence VGPTTGYF, or a CDR H3 having the sequence EVVGPTTGYF. In embodiments, CDR L3 has the amino acid sequence QSADSSFTYP. In embodiments, CDR L3 has the amino acid sequence QSA. In embodiments, CDR L3 has the amino acid sequence QSAD. In embodiments, CDR L3 has the amino acid sequence QSADS. In embodiments, CDR L3 has the amino acid sequence QSADS S. In embodiments, CDR L3 has the amino acid sequence FTYP. In embodiments, CDR L3 has the amino acid sequence SFTYP. In embodiments, CDR L3 has the amino acid sequence SSFTYP. In embodiments, CDR L3 has the amino acid sequence DSSFTYP. In embodiments, CDR L3 has the amino acid sequence of SEQ ID NO:3. In embodiments, CDR H1 is as set forth in SEQ ID NO:4. In embodiments, CDR H1 is as set forth in SEQ ID NO:15. In embodiments, CDR H1 has the sequence GGFISSRNW. In embodiments, CDR H2 has the sequence WIGEVYHTGI. In embodiments, CDR H2 has the sequence EVYHTGI. In embodiments, CDR H2 has the sequence WIGEVYHTGITN. In embodiments, CDR H2 has the sequence WIGEVYHTGIT. In embodiments, CDR H2 has the sequence EVYHTGITN. In embodiments, CDR H2 has the sequence YHTGITN. In embodiments, CDR H2 has the sequence ITN. In embodiments, CDR H2 is as set forth in SEQ ID NO:5. In embodiments, CDR H2 is as set forth in SEQ ID NO:16. In embodiments, CDR H3 has the sequence EVV. In embodiments, CDR H3 has the sequence EVVG. In embodiments, CDR H3 has the sequence EVVGP. In embodiments, CDR H3 has the sequence EVVGPT. In embodiments, CDR H3 has the sequence EVVGPTT. In embodiments, CDR H3 has the sequence EVVGPTTG. In embodiments, CDR H3 has the sequence EVVGPTTGY. In embodiments, CDR H3 has the sequence VGP. In embodiments, CDR H3 has the sequence GYF. In embodiments, CDR H3 has the sequence TGYF. In embodiments, CDR H3 has the sequence TTGYF. In embodiments, CDR H3 has the sequence PTTGYF. In embodiments, CDR H3 has the sequence GPTTGYF. In embodiments, CDR H3 has the sequence VGPTTGYF. In embodiments, CDR H3 has the sequence EVVGPTTGYF. In embodiments, CDR H3 is as set forth in SEQ ID NO:6. In embodiments, the antibody further comprises a heavy chain framework region which comprises HFR2 as set forth in SEQ ID NO:12, a HFR2 as set forth in SEQ ID NO:18, or a HFR2 having the sequence WIGE. In embodiments HFR2 is as set forth in SEQ ID NO:12. In embodiments HFR2 is as set forth in SEQ ID NO:18. In embodiments HFR2 has the sequence WIGE. In embodiments, the antibody further comprises a heavy chain framework region which comprises HFR3 as set forth in SEQ ID NO:13, a HFR3 as set forth in SEQ ID NO:19, a HFR3 having the sequence ITNYNPSLKSRVTISVDKSKNQFSLKLSSVTAADTAVYYCAR, or a HFR3 having the sequence ITN. In embodiments HFR3 is as set forth in SEQ ID NO:13. In embodiments HFR3 is as set forth in SEQ ID NO:19. In embodiments HFR3 has the sequence ITN. In embodiments HFR3 has the sequence ITNYNPSLKSRVTISVDKSKNQFSLKLSSVTAADTAVYYCAR. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

The disclosure provides an antibody having a binding affinity to fentanyl that is greater than or equal to the binding affinity to fentanyl of the antibody having the light chain sequence of SEQ ID NO:22 and the heavy chain sequence of SEQ ID NO:25. In embodiments, the disclosure provides an antibody having a binding affinity to fentanyl that is 0% to 25% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 20% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 15% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 10% less than the binding affinity to fentanyl of the antibody having the light chain sequence of SEQ ID NO:22 and the heavy chain sequence of SEQ ID NO:25. In embodiments, the antibody has a binding affinity to fentanyl that is 0% to 5% less than the binding affinity to fentanyl of the antibody having the light chain sequence of SEQ ID NO:22 and the heavy chain sequence of SEQ ID NO:25. In embodiments, the binding affinity is determined by the assay set forth in Example 2. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

In embodiments, the disclosure provides an antibody having a binding affinity (K_{D}) to fentanyl of at least 10⁻⁷ M. In embodiments, the disclosure provides an antibody having a binding affinity (K_{D}) to fentanyl of at least 10⁻⁸ M. In embodiments, the disclosure provides an antibody having a binding affinity (K_{D}) to fentanyl of at least 10⁻⁹ M. In embodiments, the disclosure provides an antibody having a binding affinity (K_{D}) to fentanyl of at least 10⁻¹⁰ M. In embodiments, the disclosure provides an antibody having a binding affinity (K_{D}) to fentanyl of at least 10⁻¹¹ M. In embodiments, the disclosure provides an antibody having a binding affinity (K_{D}) to fentanyl from about 10⁻⁷ M to about 10⁻¹¹ M. In embodiments, the disclosure provides an antibody having a binding affinity (K_{D}) to fentanyl from about 10⁻⁸ M to about 10⁻¹¹ M. In embodiments, the disclosure provides an antibody having a binding affinity (K_{D}) to fentanyl from about 10⁻⁹ M to about 10⁻¹¹ M. In embodiments, the binding affinity is determined by the assay set forth in Example 2. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

In embodiments, the antibodies described herein comprises a light chain variable region. In embodiments, the light chain variable region has an amino acid sequence with at least 50%, 55%, 60%, 65%, 70%, 75%, 80%, 85%, 88%, 90%, 91%, 92%, 93%, 94%, 95%, 96%, 97%, 98%, 99%, or 100% sequence identity to SEQ ID NO:20. In embodiments, the light chain variable region has an amino acid sequence with at least 80% sequence identity to SEQ ID NO:20. In embodiments, the light chain variable region has an amino acid sequence with at least 85% sequence identity to SEQ ID NO:20. In embodiments, the light chain variable region has an amino acid sequence with at least 90% sequence identity to SEQ ID NO:20. In embodiments, the light chain variable region has an amino acid sequence with at least 92% sequence identity to SEQ ID NO:20. In embodiments, the light chain variable region has an amino acid sequence with at least 94% sequence identity to SEQ ID NO:20. In embodiments, the light chain variable region has an amino acid sequence with at least 95% sequence identity to SEQ ID NO:20. In embodiments, the light chain variable region has an amino acid sequence with at least 96% sequence identity to SEQ ID NO:20. In embodiments, the light chain variable region has an amino acid sequence with at least 98% sequence identity to SEQ ID NO:20. In embodiments, the light chain variable region is SEQ ID NO:20 and having one or more conservatively modified variants. In embodiments, the light chain variable region is SEQ ID NO:20 and having 1 to 20 conservatively modified variants. In embodiments, the light chain variable region is SEQ ID NO:20 and having 1 to 15 conservatively modified variants. In embodiments, the light chain variable region is SEQ ID NO:20 and having 1 to 10 conservatively modified variants. In embodiments, the light chain variable region is SEQ ID NO:20 and having 1 to 5 conservatively modified variants. As described herein, a conservatively modified variant includes a conservative substitution, deletion of an amino acid, addition of an amino acid, or a combination of two or more thereof. In embodiments, a conservatively modified variation is a conservative substitution. In embodiments, a conservatively modified variation is a deletion of an amino acid. In embodiments, a conservatively modified variation is an addition of an amino acid. In embodiments, the light chain variable region comprises SEQ ID NO:20. In embodiments, the light chain constant region is as set forth in SEQ ID NO:20. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

In embodiments, the antibodies described herein comprises a heavy chain variable region. In embodiments, the heavy chain variable region has an amino acid sequence with at least 50%, 55%, 60%, 65%, 70%, 75%, 80%, 85%, 88%, 90%, 91%, 92%, 93%, 94%, 95%, 96%, 97%, 98%, 99%, or 100% sequence identity to SEQ ID NO:23. In embodiments, the heavy chain variable region has an amino acid sequence with at least 80% sequence identity to SEQ ID NO:23. In embodiments, the heavy chain variable region has an amino acid sequence with at least 85% sequence identity to SEQ ID NO:23. In embodiments, the heavy chain variable region has an amino acid sequence with at least 90% sequence identity to SEQ ID NO:23. In embodiments, the heavy chain variable region has an amino acid sequence with at least 92% sequence identity to SEQ ID NO:23. In embodiments, the heavy chain variable region has an amino acid sequence with at least 94% sequence identity to SEQ ID NO:23. In embodiments, the heavy chain variable region has an amino acid sequence with at least 95% sequence identity to SEQ ID NO:23. In embodiments, the heavy chain variable region has an amino acid sequence with at least 96% sequence identity to SEQ ID NO:23. In embodiments, the heavy chain variable region has an amino acid sequence with at least 98% sequence identity to SEQ ID NO:23. In embodiments, the heavy chain variable region is SEQ ID NO:23 and having one or more conservatively modified variants. In embodiments, the heavy chain variable region is SEQ ID NO:23 and having 1 to 20 conservatively modified variants. In embodiments, the heavy chain variable region is SEQ ID NO:23 and having 1 to 15 conservatively modified variants. In embodiments, the heavy chain variable region is SEQ ID NO:23 and having 1 to 10 conservatively modified variants. In embodiments, the heavy chain variable region is SEQ ID NO:23 and having 1 to 5 conservatively modified variants. As described herein, a conservatively modified variant includes a conservative substitution, deletion of an amino acid, addition of an amino acid, or a combination of two or more thereof. In embodiments, a conservatively modified variation is a conservative substitution. In embodiments, a conservatively modified variation is a deletion of an amino acid. In embodiments, a conservatively modified variation is an addition of an amino acid. In embodiments, the heavy chain variable region comprises SEQ ID NO:23. In embodiments, the heavy chain constant region is as set forth in SEQ ID NO:23. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

In embodiments, the antibodies described herein comprises a light chain constant region. In embodiments, the light chain constant region has an amino acid sequence with at least 50%, 55%, 60%, 65%, 70%, 75%, 80%, 85%, 88%, 90%, 91%, 92%, 93%, 94%, 95%, 96%, 97%, 98%, 99%, or 100% sequence identity to SEQ ID NO:21. In embodiments, the light chain constant region has an amino acid sequence with at least 80% sequence identity to SEQ ID NO:21. In embodiments, the light chain constant region has an amino acid sequence with at least 85% sequence identity to SEQ ID NO:21. In embodiments, the light chain constant region has an amino acid sequence with at least 90% sequence identity to SEQ ID NO:21. In embodiments, the light chain constant region has an amino acid sequence with at least 92% sequence identity to SEQ ID NO:21. In embodiments, the light chain constant region has an amino acid sequence with at least 94% sequence identity to SEQ ID NO:21. In embodiments, the light chain constant region has an amino acid sequence with at least 95% sequence identity to SEQ ID NO:21. In embodiments, the light chain constant region has an amino acid sequence with at least 96% sequence identity to SEQ ID NO:21. In embodiments, the light chain constant region has an amino acid sequence with at least 98% sequence identity to SEQ ID NO:21. In embodiments, the light chain constant region is SEQ ID NO:21 and having one or more conservatively modified variants. In embodiments, the light chain constant region is SEQ ID NO:21 and having 1 to 20 conservatively modified variants. In embodiments, the light chain constant region is SEQ ID NO:21 and having 1 to 21 conservatively modified variants. In embodiments, the light chain constant region is SEQ ID NO:21 and having 1 to 10 conservatively modified variants. In embodiments, the light chain constant region is SEQ ID NO:21 and having 1 to 5 conservatively modified variants. As described herein, a conservatively modified variant includes a conservative substitution, deletion of an amino acid, addition of an amino acid, or a combination of two or more thereof. In embodiments, a conservatively modified variation is a conservative substitution. In embodiments, a conservatively modified variation is a deletion of an amino acid. In embodiments, a conservatively modified variation is an addition of an amino acid. In embodiments, the light chain constant region comprises SEQ ID NO:21. In embodiments, the light chain constant region is as set forth in SEQ ID NO:21. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof.

In embodiments, the antibodies described herein comprises a heavy chain constant region. In embodiments, the heavy chain constant region has an amino acid sequence with at least 50%, 55%, 60%, 65%, 70%, 75%, 80%, 85%, 88%, 90%, 91%, 92%, 93%, 94%, 95%, 96%, 97%, 98%, 99%, or 100% sequence identity to SEQ ID NO:24. In embodiments, the heavy chain constant region has an amino acid sequence with at least 80% sequence identity to SEQ ID NO:24. In embodiments, the heavy chain constant region has an amino acid sequence with at least 85% sequence identity to SEQ ID NO:24. In embodiments, the heavy chain constant region has an amino acid sequence with at least 90% sequence identity to SEQ ID NO:24. In embodiments, the heavy chain constant region has an amino acid sequence with at least 92% sequence identity to SEQ ID NO:24. In embodiments, the heavy chain constant region has an amino acid sequence with at least 94% sequence identity to SEQ ID NO:24. In embodiments, the heavy chain constant region has an amino acid sequence with at least 95% sequence identity to SEQ ID NO:24. In embodiments, the heavy chain constant region has an amino acid sequence with at least 96% sequence identity to SEQ ID NO:24. In embodiments, the heavy chain constant region has an amino acid sequence with at least 98% sequence identity to SEQ ID NO:24. In embodiments, the light chain constant region is SEQ ID NO:24 and having one or more conservatively modified variants. In embodiments, the light chain constant region is SEQ ID NO:24 and having 1 to 20 conservatively modified variants. In embodiments, the light chain constant region is SEQ ID NO:24 and having 1 to 15 conservatively modified variants. In embodiments, the light chain constant region is SEQ ID NO:24 and having 1 to 10 conservatively modified variants. In embodiments, the light chain constant region is SEQ ID NO:24 and having 1 to 5 conservatively modified variants. As described herein, a conservatively modified variant includes a conservative substitution, deletion of an amino acid, addition of an amino acid, or a combination of two or more thereof. In embodiments, a conservatively modified variation is a conservative substitution. In embodiments, a conservatively modified variation is a deletion of an amino acid. In embodiments, a conservatively modified variation is an addition of an amino acid. In embodiments, the heavy chain constant region comprises SEQ ID NO:24. In embodiments, the heavy chain constant region is as set forth in SEQ ID NO:24. In embodiments, the antibody is an IgG antibody. In embodiments, the antibody is an IgG1 antibody. In embodiments, the antibody is a Fab′ fragment. In embodiments, the antibody is a F(ab)′₂ fragment. In embodiments, the antibody is a scFv. In embodiments, the antibody is a humanized antibody. In embodiments, the antibody is a chimeric antibody. In embodiments, the antibody is a monoclonal antibody. Provided herein is a nucleic acid capable of encoding the antibody described herein, including all embodiments thereof. Provided herein is a vector comprising a nucleic acid capable of encoding the antibody described herein, including all embodiments thereof

Complexes

Provided herein are complexes comprising an antibody described herein (including aspects and embodiments thereof) and an opioid. As described herein, the antibodies of the disclosure bind to fentanyl, a fentanyl analogue, carfentanil, or a carfentanil analogue, thereby forming a complex of the antibody and the opioid. In embodiments, the disclosure provides an antibody described herein (including aspects and embodiments thereof) and a synthetic opioid. In embodiments, the disclosure provides an antibody described herein (including aspects and embodiments thereof) and fentanyl, a fentanyl analogue, carfentanil, or a carfentanil analogue. In embodiments, the disclosure provides an antibody described herein (including aspects and embodiments thereof) and fentanyl. In embodiments, the disclosure provides an antibody described herein (including aspects and embodiments thereof) and carfentanil. In embodiments, the disclosure provides an antibody described herein (including aspects and embodiments thereof) and a fentanyl analogue. In embodiments, the disclosure provides an antibody described herein (including aspects and embodiments thereof) and a carfentanil analogue. In aspects, the fentanyl analogue is acetylfentanyl, alfentanil, butylfentanyl, butyrfentanyl, para-tolylfentanyl, 3-methylfentanyl, α-methylfentanyl, remifentanil, mefentanyl, phenaridine, ohmefentanyl, or mirfentanil. In aspects, the carfentanil analogue is sufentanil, remifentanil, alfentanil, lofentanil, brifentanil, trefentanil

Nucleic Acids

Provided herein are isolated nucleic acids encoding an antibody provided herein including embodiments and aspects thereof. For example, the nucleic acid may encode at least one CDR, specific residues involved in binding the epitope, or binding framework residues.

In embodiments, the nucleic acid has at least 75% sequence identity to SEQ ID NO:26. In embodiments, the nucleic acid has at least 80% sequence identity to SEQ ID NO:26. In embodiments, the nucleic acid has at least 85% sequence identity to SEQ ID NO:26. In embodiments, the nucleic acid has at least 90% sequence identity to SEQ ID NO:26. In embodiments, the nucleic acid has at least 92% sequence identity to SEQ ID NO:26. In embodiments, the nucleic acid has at least 94% sequence identity to SEQ ID NO:26. In embodiments, the nucleic acid has at least 95% sequence identity to SEQ ID NO:26. In embodiments, the nucleic acid has at least 96% sequence identity to SEQ ID NO:26. In embodiments, the nucleic acid has at least 98% sequence identity to SEQ ID NO:26. In embodiments, the nucleic acid comprises SEQ ID NO:26. In embodiments, the nucleic acid is as set forth in SEQ ID NO:26. Provided herein is a vector comprising a nucleic acid capable of encoding the antibody described herein, including all embodiments thereof.

In embodiments, the nucleic acid has at least 75% sequence identity to SEQ ID NO:27. In embodiments, the nucleic acid has at least 80% sequence identity to SEQ ID NO:27. In embodiments, the nucleic acid has at least 85% sequence identity to SEQ ID NO:27. In embodiments, the nucleic acid has at least 90% sequence identity to SEQ ID NO:27. In embodiments, the nucleic acid has at least 92% sequence identity to SEQ ID NO:27. In embodiments, the nucleic acid has at least 94% sequence identity to SEQ ID NO:27. In embodiments, the nucleic acid has at least 95% sequence identity to SEQ ID NO:27. In embodiments, the nucleic acid has at least 96% sequence identity to SEQ ID NO:27. In embodiments, the nucleic acid has at least 98% sequence identity to SEQ ID NO:27. In embodiments, the nucleic acid comprises SEQ ID NO:27. In embodiments, the nucleic acid is as set forth in SEQ ID NO:27. Provided herein is a vector comprising a nucleic acid capable of encoding the antibody described herein, including all embodiments thereof.

In embodiments, the nucleic acid has at least 75% sequence identity to SEQ ID NO:28. In embodiments, the nucleic acid has at least 80% sequence identity to SEQ ID NO:28 In embodiments, the nucleic acid has at least 85% sequence identity to SEQ ID NO:28. In embodiments, the nucleic acid has at least 90% sequence identity to SEQ ID NO:28. In embodiments, the nucleic acid has at least 92% sequence identity to SEQ ID NO:28. In embodiments, the nucleic acid has at least 94% sequence identity to SEQ ID NO:28. In embodiments, the nucleic acid has at least 95% sequence identity to SEQ ID NO:28. In embodiments, the nucleic acid has at least 96% sequence identity to SEQ ID NO:28. In embodiments, the nucleic acid has at least 98% sequence identity to SEQ ID NO:28. In embodiments, the nucleic acid comprises SEQ ID NO:28. In embodiments, the nucleic acid is as set forth in SEQ ID NO:28. Provided herein is a vector comprising a nucleic acid capable of encoding the antibody described herein, including all embodiments thereof

Methods of Treatment

In aspects, the disclosure provides methods of treating an opioid overdose in a patient in need thereof by administering to the patient an effective amount of an antibody described herein. In aspects, the disclosure provides methods of treating an opioid overdose in a patient in need thereof by administering to the patient an effective amount of a pharmaceutical composition comprising an antibody described herein and a pharmaceutically acceptable excipient. The methods of treating an opioid overdose can alternatively be referred to as methods for reversing an opioid overdose, whereby the antibodies described herein are administered to a patient who is experiencing an opioid overdose. In aspects, the opioid is fentanyl, a fentanyl analogue, carfentanil, a carfentanil analogue, or a combination of two or more thereof. In aspects, the opioid is fentanyl or a fentanyl analogue. In aspects, the opioid is fentanyl. In aspects, the opioid is a fentanyl analogue. In aspects, the opioid is carfentanil or a carfentanil analogue. In aspects, the opioid is a carfentanil analogue.

In aspects, the disclosure provides methods of preventing an opioid overdose in a patient in need thereof by administering to the patient an effective amount of an antibody described herein. In aspects, the disclosure provides methods of preventing an opioid overdose in a patient in need thereof by administering to the patient an effective amount of a pharmaceutical composition comprising an antibody described herein and a pharmaceutically acceptable excipient. In aspects, the opioid is fentanyl, a fentanyl analogue, carfentanil, a carfentanil analogue, or a combination of two or more thereof. In aspects, the opioid is fentanyl or a fentanyl analogue. In aspects, the opioid is fentanyl. In aspects, the opioid is a fentanyl analogue. In aspects, the opioid is carfentanil or a carfentanil analogue. In aspects, the opioid is a carfentanil analogue.

In aspects, the disclosure provides methods of treating opioid use disorder in a patient in need thereof by administering to the patient an effective amount of an antibody described herein. In aspects, the disclosure provides methods of treating opioid use disorder in a patient in need thereof by administering to the patient an effective amount of a pharmaceutical composition comprising an antibody described herein and a pharmaceutically acceptable excipient. In aspects, the opioid is fentanyl, a fentanyl analogue, carfentanil, a carfentanil analogue, or a combination of two or more thereof. In aspects, the opioid is fentanyl or a fentanyl analogue. In aspects, the opioid is fentanyl. In aspects, the opioid is a fentanyl analogue. In aspects, the opioid is carfentanil or a carfentanil analogue. In aspects, the opioid is a carfentanil analogue.

In embodiments, the disclosure provides methods of treating opioid-induced respiratory depression by administering to a patient an effective amount of an antibody described herein. In embodiments, the disclosure provides methods of treating opioid-induced respiratory depression by administering to a patient an effective amount of a pharmaceutical composition comprising an antibody described herein and a pharmaceutically acceptable excipient. The methods may alternatively be referred to as methods of reducing respiratory depression caused by an opioid. n aspects, the opioid is fentanyl, a fentanyl analogue, carfentanil, a carfentanil analogue, or a combination of two or more thereof. In aspects, the opioid is fentanyl or a fentanyl analogue. In aspects, the opioid is fentanyl. In aspects, the opioid is a fentanyl analogue. In aspects, the opioid is carfentanil or a carfentanil analogue. In aspects, the opioid is a carfentanil analogue.

In embodiments, the disclosure provides methods of preventing opioid-induced respiratory depression by administering to a patient an effective amount of an antibody described herein. In embodiments, the disclosure provides methods of preventing opioid-induced respiratory depression by administering to a patient an effective amount of a pharmaceutical composition comprising an antibody described herein and a pharmaceutically acceptable excipient. In aspects, the opioid is fentanyl, a fentanyl analogue, carfentanil, a carfentanil analogue, or a combination of two or more thereof. In aspects, the opioid is fentanyl or a fentanyl analogue. In aspects, the opioid is fentanyl. In aspects, the opioid is a fentanyl analogue. In aspects, the opioid is carfentanil or a carfentanil analogue. In aspects, the opioid is a carfentanil analogue.

As used herein, the term “administering” means oral administration, administration as a suppository, topical contact, intravenous, parenteral, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

The terms “treating”, or “treatment” refers to any indicia of success in the therapy or amelioration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

With reference to the Diagnostic and Statistical Manual for Mental Disorders, 5th Edition, American Psychiatric Association, 2013 (also referred to herein as DSMS), the disclosure of which is incorporated by reference herein in its entirety, “opioid use disorder” is characterized by signs and symptoms that reflect compulsive, prolonged self-administration of opioid substances that are used for no legitimate medical purpose or, if another medical condition is present that requires opioid treatment, they are used in doses greatly in excess of the amount needed for that medical condition. In aspects, the opioid use disorder is moderate opioid use disorder. “Moderate opioid use disorder” is defined by reference to the DSMS Opioid Use Disorder Checklist (ICD-9-CM code 304.00 or ICD-10-CM code F11.20) as having the presence of 4 or 5 symptoms indicated in the DSMS Opioid Use Disorder Checklist. In aspects, the opioid use disorder is severe opioid use disorder. “Severe opioid use disorder” is defined by reference to the DSMS Opioid Use Disorder Checklist (ICD-9-CM code 304.00 or ICD-10-CM code F11.20) as having the presence of 6 or more symptoms indicated in the DSMS Opioid Use Disorder Checklist. In aspects, the opioid use disorder is moderate-to-severe opioid use disorder. Moderate-to-severe opioid use disorder refers to the presence of 4 or more symptoms indicated in the DSMS Opioid Use Disorder Checklist. In aspects, the opioid use disorder is mild opioid use disorder. “Mild opioid use disorder” is defined by reference to the DSMS Opioid Use Disorder Checklist (ICD-9-CM code 305.50 or ICD-10-CM code F11.10) as having the presence of 2 or 3 symptoms indicated in the DSMS Opioid Use Disorder Checklist. In aspects, the opioid use disorder is mild-to-moderate opioid use disorder. Mild-to-moderate opioid use disorder refers to the presence of 2 to 5 symptoms indicated in the DSMS Opioid Use Disorder Checklist. In aspects, “treating opioid use disorder” encompasses one or more of: (i) reducing opioid withdrawal symptoms, (ii) eliminating opioid withdrawal symptoms, (iii) reducing opioid craving, (iv) eliminating opioid craving, (v) reducing illicit opioid use, (vi) eliminating illicit opioid use, and (vii) inducing opioid abstinence. The term “opioid use disorder” can be interchangeably used with the terms “opioid addiction” or “opioid dependence.”

Compositions

Provided herein are compositions comprising the antibodies described herein (including embodiments and aspects thereof) and a pharmaceutically acceptable excipient.

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such compositions can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the antibodies of the disclosure. One of skill in the art will recognize that other pharmaceutical excipients are useful.

Solutions of the antibodies can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these compositions can contain a preservative to prevent the growth of microorganisms.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the antibodies in the required amount in the appropriate solvent followed by filtered sterilization. Generally, dispersions are prepared by incorporating the antibodies into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The composition of more, or highly, concentrated solutions for direct injection is also contemplated. Solvents, such as dimethyl sulfoxide, can be used for extremely rapid penetration, delivering high concentrations of the active agents to a small area.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic compositions and appropriate drug stabilizers, if required, may be included in the formulation. Various commercial nasal compositions are known.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In aspects, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the antibodies may be incorporated with excipients and used in the form of ingestible tablets, buccal tablets, troches, capsules, elixirs, suspensions, syrups, wafers, and the like. The percentage of the compositions may, of course, be varied and may conveniently be between about 1 to about 75% of the weight of the unit. The amount of antibodies in such compositions is such that a suitable dosage can be obtained.

The formulations of antibodies can be presented in unit-dose or multi-dose sealed containers, such as ampules and vials. Thus, the composition can be in unit dosage form. In such form the composition is subdivided into unit doses containing appropriate quantities of antibodies. Thus, the compositions can be administered in a variety of unit dosage forms depending upon the method of administration.

Dosages

Pharmaceutical compositions include compositions wherein the active ingredient (i.e., antibody) is contained in a therapeutically effective amount, i.e., in an amount effective to achieve its intended purpose. The actual amount effective for a particular application will depend, inter alia, on the condition being treated, as judged by a practitioner in the medical arts.

A “effective amount” is an amount sufficient for an antibody to accomplish a stated purpose relative to the absence of the compound (e.g. achieve the effect for which it is administered, treat a disease, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of an antibody is an amount of that antibody, when administered to a subject, will have the intended prophylactic effect, e.g., preventing or delaying the onset (or reoccurrence) of a disease, pathology or condition, or reducing the likelihood of the onset (or reoccurrence) of a disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any antibody described herein, the therapeutically effective amount can be initially determined from cell culture assays. Target concentrations will be those concentrations of antibody that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages and frequency (single or multiple doses) of the antibodies may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the antibody. Thereafter, the dosage is increased by small increments until the optimum effect under circumstances is reached. Dosage amounts and intervals can be adjusted individually to provide levels of the antibody effective for the particular clinical indication being treated. This will provide a therapeutic regimen that is commensurate with the severity of the individual's disease state. In embodiments, the antibody is administered at an amount from about 0.001 μg to about 10,000 μg.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of monoclonal antibodies by considering factors such as compound potency, relative bioavailability, patient body weight, presence and severity of adverse side effects.

### Embodiments 1-31

Embodiment 1. An antibody comprising: (i) a light chain variable region which comprises a CDR L1 having an amino acid sequence with at least 95% sequence identity to SEQ ID NO:1, a CDR L2 having an amino acid sequence with at least 95% sequence identity to SEQ ID NO:2, and a CDR L3 having an amino acid sequence with at least 95% sequence identity to SEQ ID NO:3; and (ii) a heavy chain variable region which comprises: a CDR H1 having an amino acid sequence with at least 95% sequence identity to SEQ ID NO:15, a CDR H2 having an amino acid sequence with at least 95% sequence identity to SEQ ID NO:16, and a CDR H3 having an amino acid sequence with at least 95% sequence identity to SEQ ID NO:6.
Embodiment 2. The antibody of Embodiment 1, wherein: (i) the light chain variable region comprises CDR L1 as set forth in SEQ ID NO:1, CDR L2 as set forth in SEQ ID NO:2, and CDR L3 as set forth in SEQ ID NO:3; and (ii) the heavy chain variable region comprises CDR H1 as set forth in SEQ ID NO:15, CDR H2 as set forth in SEQ ID NO:16, and CDR H3 as set forth in SEQ ID NO:6.
Embodiment 3. The antibody of Embodiment 1 or 2, further comprising a light chain framework region which comprises a LFR1 having an amino acid sequence with at least 95% sequence identity to SEQ ID NO:7, a LFR2 having an amino acid sequence with at least 95% sequence identity to SEQ ID NO:8, a LFR3 having an amino acid sequence with at least 95% sequence identity to SEQ ID NO:9, and a LFR4 having an amino acid sequence with at least 95% sequence identity to SEQ ID NO:10.
Embodiment 4. The antibody of Embodiment 3, wherein the light chain framework region comprises LFR1 as set forth in SEQ ID NO:7, LFR2 as set forth in SEQ ID NO:8, LFR3 as set forth in SEQ ID NO:9, and LFR4 as set forth in SEQ ID NO:10.
Embodiment 5. The antibody of any one of Embodiments 1 to 4, further comprising a heavy chain framework region which comprises a HFR1 having an amino acid sequence with at least 95% sequence identity to SEQ ID NO:17; a HFR2 having an amino acid sequence with at least 95% sequence identity to SEQ ID NO:18, a HFR3 having an amino acid sequence with at least 95% sequence identity to SEQ ID NO:19, and a HFR4 having an amino acid sequence with at least 95% sequence identity to SEQ ID NO:14.
Embodiment 6. The antibody of Embodiment 5, wherein the heavy chain framework region comprises HFR1 as set forth in SEQ ID NO:17; HFR2 as set forth in SEQ ID NO:18, HFR3 as set forth in SEQ ID NO:19, and HFR4 as set forth in SEQ ID NO:14.
Embodiment 7. An antibody comprising: (i) a light chain variable region which comprises a CDR L1 having an amino acid sequence with at least 95% sequence identity to SEQ ID NO:1, a CDR L2 having an amino acid sequence with at least 95% sequence identity to SEQ ID NO:2, and a CDR L3 having an amino acid sequence with at least 95% sequence identity to SEQ ID NO:3; and (ii) a heavy chain variable region which comprises a CDR H1 having an amino acid sequence with at least 95% sequence identity to SEQ ID NO:4, a CDR H2 having an amino acid sequence with at least 95% sequence identity to SEQ ID NO:5, and a CDR H3 having an amino acid sequence with at least 95% sequence identity to SEQ ID NO:6.
Embodiment 8. The antibody of Embodiment 7, wherein: (i) the light chain variable region which comprises CDR L1 as set forth in SEQ ID NO:1, CDR L2 as set forth in SEQ ID NO:2, and CDR L3 as set forth in SEQ ID NO:3; and (ii) the heavy chain variable region which comprises CDR H1 as set forth in SEQ ID NO:4, CDR H2 as set forth in SEQ ID NO:5, and CDR H3 as set forth in SEQ ID NO:6.
Embodiment 9. The antibody of Embodiment 7 or 8, further comprising a light chain framework region which comprises a LFR1 having an amino acid sequence with at least 95% sequence identity to SEQ ID NO:7, a LFR2 having an amino acid sequence with at least 95% sequence identity to SEQ ID NO:8, a LFR3 having an amino acid sequence with at least 95% sequence identity to SEQ ID NO:9, and a LFR4 having an amino acid sequence with at least 95% sequence identity to SEQ ID NO:10.
Embodiment 10. The antibody of Embodiment 9, wherein the light chain framework region comprises LFR1 as set forth in SEQ ID NO:7, LFR2 as set forth in SEQ ID NO:8, LFR3 as set forth in SEQ ID NO:9, and LFR4 as set forth in SEQ ID NO:10.
Embodiment 11. The antibody of any one of Embodiments 7 to 10, further comprising a heavy chain framework region which comprises a HFR1 having an amino acid sequence with at least 95% sequence identity to SEQ ID NO:11; a HFR2 having an amino acid sequence with at least 95% sequence identity to SEQ ID NO:12, a HFR3 having an amino acid sequence with at least 95% sequence identity to SEQ ID NO:13, and a HFR4 having an amino acid sequence with at least 95% sequence identity to SEQ ID NO:14.
Embodiment 12. The antibody of Embodiment 11, wherein the heavy chain framework region comprises HFR1 as set forth in SEQ ID NO:11; HFR2 as set forth in SEQ ID NO:12, HFR3 as set forth in SEQ ID NO:13, and HFR4 as set forth in SEQ ID NO:14.
Embodiment 13. An antibody comprising a light chain variable region having at least 85% sequence identity to SEQ ID NO:20 and a heavy chain variable region having at least 85% sequence identity to SEQ ID NO:23.
Embodiment 14. The antibody of Embodiment 13, wherein the light chain variable region comprises SEQ ID NO:20 and the heavy chain variable region comprises SEQ ID NO:23.
Embodiment 15. The antibody of any one of Embodiments 1 to 14, further comprising a light chain constant region having an amino acid sequence with at least 85% identity to SEQ ID NO:21.
Embodiment 16. The antibody of Embodiment 15, wherein the light chain constant region comprises SEQ ID NO:21.
Embodiment 17. The antibody of any one of Embodiments 1 to 16, further comprising a heavy chain constant region with at least 85% identity to SEQ ID NO:24.
Embodiment 18. The antibody of Embodiment 17, wherein the heavy chain constant region comprises SEQ ID NO:24.
Embodiment 19. The antibody of any one of Embodiments 1 to 18, wherein the antibody is an IgG antibody.
Embodiment 20. The antibody of Embodiment 19, wherein the IgG antibody is an IgG1 antibody.
Embodiment 21. The antibody of any one of Embodiments 1 to 20, wherein the antibody is a Fab′ fragment or a F(ab)′₂ fragment.
Embodiment 22. The antibody of any one of Embodiments 1 to 19, wherein the antibody is a single chain variable fragment.
Embodiments 23. The antibody of any one of Embodiments 1 to 22, wherein the antibody is a humanized monoclonal antibody.
Embodiments 24. A pharmaceutical composition comprising the antibody of any one of Embodiments 1 to 23 and a pharmaceutically acceptable excipient.
Embodiment 25. A method of treating or preventing an opioid overdose in a patient in need thereof, the method comprising administering to the patient an effective amount of the antibody of any one of Embodiments 1 to 23 or the pharmaceutical composition of Embodiment 24.
Embodiment 26. A method of treating opioid use disorder in a patient in need thereof, the method comprising administering to the patient an effective amount of the antibody of any one of Embodiments 1 to 23 or the pharmaceutical composition of Embodiment 24.
Embodiment 27. A method of treating or preventing opioid-induced respiratory depression in a patient in need thereof, the method comprising administering to the patient an effective amount of the antibody of any one of Embodiments 1 to 23 or the pharmaceutical composition of Embodiment 24.
Embodiment 28. An isolated nucleic acid encoding the antibody of any one of Embodiments 1 to 23.
Embodiment 29. An isolated nucleic acid having a nucleic acid sequence with at least 85% sequence identity to SEQ ID NO:26, SEQ ID NO:27, or SEQ ID NO:28.
Embodiment 30. A vector comprising a nucleic acid encoding the antibody of any one of Embodiments 1 to 23.
Embodiment 31. A complex comprising: (i) the antibody of any one of Embodiments 1 to 23; and (ii) fentanyl, a fentanyl analogue, carfentanil, or a carfentanil analogue.

### EXAMPLES

The anti-opioid mAbs described herein possess high binding affinity for fentanyl, carfentanil, and related analogues, thus they are able to effectively mitigate the harmful effects of synthetic opioids. The mAbs could be infused into patients for immediate treatment of opioid overdose or to protect them from subsequent exposures to opioids. In the latter case, patients would gain protection from not only the acutely lethal effects of high dose fentanyl, but also the addictive effects of low dose fentanyl. Therefore, an anti-opioid mAb can be used as an antidote to synthetic opioids as well as a therapy for opioid use disorder.

The high degree of potency of fentanyl and other synthetic opioids as well as their long duration of action have made current opioid overdose treatments, such as naloxone, less effective. Anti-opioid mAbs are highly effective at combatting opioid intoxication because they possess picomolar affinity for the target opioids, thus they can sequester the drug in peripheral blood to prevent drug access to the sites of action in the brain. Furthermore, the mAbs do not bind to any endogenous targets in the body, granting them a highly favorable safety profile. IgG human antibodies generally show a duration of action of one month; therefore, anti-opioid mAbs could protect patients from opioid intoxication for an extended period of time that far exceeds current pharmacological therapies.

### Example 1

The antibodies described herein were made by immunizing rats with a carfentanil conjugate vaccine, selecting/sequencing B-cells with a combination of carfentanil and fentanyl biotin probes, and reengineering the antibodies with phage display libraries containing human antibody sequences. The methods are described, for example, in WO 2017/127390, WO 2020/018596, and Smith et al, “Monoclonal Antibodies for Combating Synthetic Opioid Intoxication,” J. Am. Chem. Soc., 141(26):10489-10503 (2019).

### Example 2

The binding kinetics for selected mAbs and fentanyl and its derivatives were determined by SPR using a Biacore 8K instrument (GE Healthcare Life Sciences) equipped with a Series S CMS sensor chip. Selected mAbs were immobilized into individual channels on a sensor chip surface using Amine Coupling Kit as follows. The flow cell 2 surface of each channel was activated for 7 minutes with a 1:1 mixture of 0.1 M NHS and 0.4 M EDC at a flow rate of 10 μL/min; whereas flow cell 1 of each channel was not activated. Selected mAbs resuspended in 10 mM sodium acetate (pH 5.5) were injected over activated Fc2 for 120 seconds at 10 μL/min in each channel separately. All flow cell surfaces were blocked with a 7 minute injection of 1.0 M ethanolamine-HCl (pH 8.5) at a flow rate of 10 μL/min. All assays were conducted at a flow rate of 30 μL/min at 25° C. and 37° C., using 1× PBS-P+ buffer (28-9950-84, GE Healthcare Life Sciences) as running buffer. To determine the binding kinetics, a predefined single-cycle kinetics method (SCK, Biacore 8K control software ver. 1.1.1.7442) was used with four analyte concentrations as follows: 1) 4×startup cycles (each cycle includes 300 sec of running buffer injection and 600 sec of dissociation, all at a flow rate of 30 μL/min, the chip surface was regenerated with Gly-HCl (pH 1.5) for 30 sec) were conducted before SCK analysis. 2) For SCK analysis, fentanyl and carfentanil or related analogs were prepared in running buffer at 1.25, 5, 20 and 80 nM. The compound dilutions were injected for 120 sec consecutively followed by 10800 sec of dissociation in running buffer. 3) The sensor chip surface was regenerated with 30 sec of injection of Gly-HCl (pH 1.5) solution before next cycle of SCK analysis. A blank running buffer injection was also conducted before each compound run using exactly the same conditions for SCK analysis.

All data were collected by Biacore control software in a result file. The run data sets stored in the result file were then analyzed by Biacore 8K evaluation software (ver. 1.1.1.7442) using predefined fragment/LWM single-cycle kinetics method. Each SCK analysis data set was double referenced with signal from reference Fc1 and blank running buffer injection and was fitted using a 1:1 binding model. Table 1 shows the structures of fentanyl, carfentanil, and related analogs. Tables 2A-2C show the direct binding kinetics of immobilized mAbs to free fentanyl compounds.

TABLE 1

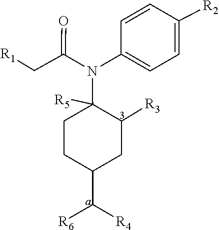

| | R¹ | R² | R³ | R⁴ | R⁵ | R⁶ |
|---|---|---|---|---|---|---|
| Fentanyl Hapten | (CH₂)₂COOH | H | H | H | H | Benzyl |
| Carfentanil Hapten | (CH₂)₂COOH | H | H | H | COOCH₃ | Benzyl |
| Fentanyl | —CH₃ | H | H | H | H | Benzyl |
| Carfentanil | —CH₃ | H | H | H | COOCH₃ | Benzyl |
| (Carfent) | | | | | | |
| 3-methylfentanyl | —CH₃ | H | —CH₃ | H | H | Benzyl |
| (3-Methyl-Fen) | | | | | | |
| α-methylfentanyl | —CH₃ | H | H | —CH₃ | H | Benzyl |
| (α-Methyl-Fen) | | | | | | |
| Acetylfentanyl | H | H | H | H | H | Benzyl |
| (Acetyl-Fen) | | | | | | |
| Butylfentanyl | —(CH₂)₂CH₃ | H | H | H | H | Benzyl |
| (ButylFen) | | | | | | |
| Tolylfentanyl | —CH₃ | —CH₃ | H | H | H | Benzyl |
| (TolylFen) | | | | | | |
| Alfentanil | H | H | H | H | CH₂OCH₃ | 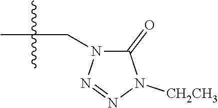 [SMILES: *Cn1nnn(CC)c1=O] |
| Remifentanil | H | H | H | H | CH₂OOCH₃ | CH₂OOCH₃ |

TABLE 2A
Head-to-head comparison of K_{D} values of Antibodies.

| | JBZ-3 | JBZ-3 | JBZ-2 | JBZ-2 | JBZ-1 | JBZ-1 | | |
|---|---|---|---|---|---|---|---|---|
| Opioid | batch1 | batch2 | batch1 | batch2 | batch1 | batch2 | P2B5 | P2C1 |
| 3- | 5.11E−09 | 2.68E−09 | 4.52E−09 | 1.42E−08 | 1.19E−09 | 1.31E−10 | 1.04E−08 | 1.42E−08 |
| Methyl- | | | | | | | | |
| Fen | | | | | | | | |
| Acetyl- | 1.88E−10 | 1.63E−10 | 3.02E−10 | 2.53E−10 | 8.43E−11 | 4.78E−11 | 1.97E−11 | 4.32E−10 |
| Fen | | | | | | | | |
| α- | 5.94E−10 | 7.59E−11 | 4.13E−09 | 7.29E−10 | 1.64E−10 | 1.32E−10 | 5.11E−10 | 1.33E−09 |
| Methyl- | | | | | | | | |
| Fen | | | | | | | | |
| ButylFen | 8.56E−11 | 9.06E−11 | 1.45E−10 | 1.39E−10 | 3.58E−11 | 1.62E−11 | 8E−12 | 3.21E−10 |
| Carfent | 6.93E−11 | 8.26E−11 | 1.27E−10 | 1.61E−10 | 3.77E−11 | 3.07E−11 | 4.66E−11 | 1.84E−10 |
| Fentanyl | 2.08E−10 | 1.85E−10 | 2.81E−10 | 2.68E−10 | 7.62E−11 | 4.66E−11 | 1.11E−10 | 4.67E−10 |
| TolylFen | 7.1E−10 | 8.6E−11 | 3.62E−11 | 4.75E−10 | 4.64E−11 | 2.91E−11 | 4.51E−10 | 5.41E−09 |

TABLE 2B
Fold change in K_{D} at 37° C.

| | JBZ-3 | JBZ-3 | JBZ-2 | JBZ-2 | JBZ-1 | JBZ-1 | | |
|---|---|---|---|---|---|---|---|---|
| Opioid | batch1 | batch2 | batch1 | batch2 | batch1 | batch2 | P2B5 | P2C1 |
| 3- | 3.82 | 4.63 | 5.82 | 43.38 | 2.71 | 14.73 | 1.04 | 38.24 |
| methyl- | | | | | | | | |
| Fen | | | | | | | | |
| Acetyl- | 3.04 | 1.62 | 1.81 | 3.58 | 2.82 | 3.35 | 6.04 | 1.92 |
| Fen | | | | | | | | |
| α- | 4.7 | 52.96 | 0.6 | 3.66 | 9.51 | 3.08 | 4.44 | 0.36 |
| Methyl- | | | | | | | | |
| Fen | | | | | | | | |
| Carfent | 6.02 | 12.35 | 7.06 | 5.84 | 3.95 | 14.2 | 0.78 | 9.67 |
| Fentanyl | 5.19 | 4.17 | 9.93 | 4.81 | 4.2 | 9.53 | 0.37 | 7.6 |
| Tolylfen | 1.03 | 2.74 | 9.59 | 1.89 | 2.84 | 2.92 | 4.75 | 0.38 |

TABLE 2C
Carfentanil IC50 Curves by SPR for Antibodies.

| | Antibodies and corresponding SPR response | | | | | |
|---|---|---|---|---|---|---|
| Carfentanil | units (RU) | | | | | |
| (nM) | JBZ-4 | JBZ-5 | JBZ-6 | JBZ-1 | JBZ-2 | JBZ-3 |
| 62.50 | 3.06 | 8.88 | 8.60 | 8.28 | 8.59 | 7.88 |
| 31.25 | 7.61 | 9.22 | 9.12 | 8.34 | 8.88 | 8.21 |
| 15.63 | 9.35 | 9.87 | 10.05 | 8.97 | 9.53 | 10.12 |
| 7.81 | 12.19 | 9.98 | 12.97 | 10.53 | 11.47 | 11.48 |
| 3.91 | 14.42 | 13.00 | 15.08 | 14.54 | 16.78 | 21.15 |
| 1.95 | 66.34 | 52.65 | 33.93 | 148.78 | 103.84 | 122.80 |
| 0.98 | 108.55 | 115.55 | 78.62 | 185.35 | 124.95 | 133.86 |
| 0.49 | 149.64 | 153.97 | 121.46 | 211.47 | 144.16 | 148.65 |
| 0.24 | 154.11 | 155.37 | 125.16 | 212.39 | 146.72 | 149.33 |
| 0.12 | 154.40 | 156.52 | 125.99 | 213.16 | 148.42 | 149.44 |
| 0.06 | 157.93 | 157.79 | 126.92 | 212.98 | 147.95 | 151.27 |
| 0.01 | 154.03 | 158.12 | 126.49 | 212.76 | 148.09 | 149.61 |
| IC50 (nM) | 1.503 | 1.397 | 1.146 | 2.275 | 2.313 | 2.568 |

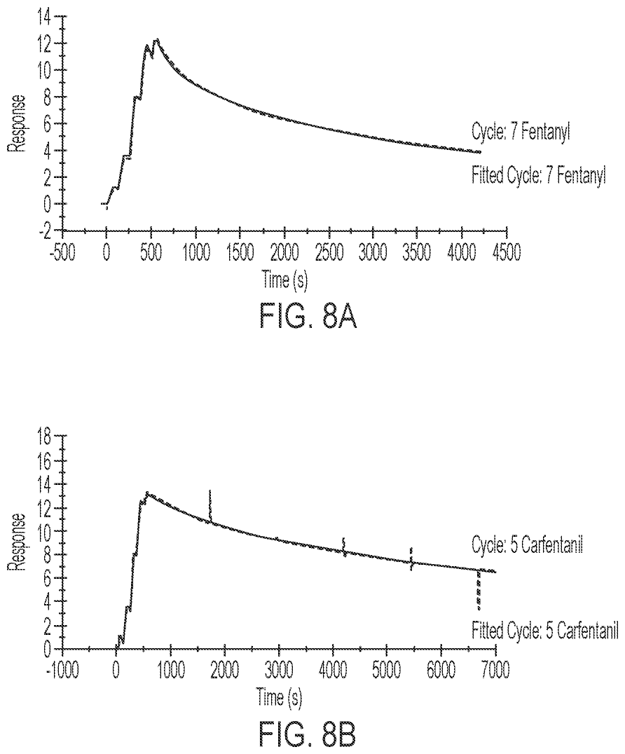

The direct binding kinetics of JBZ-4 to fentanyl and related analogues was thoroughly characterized by surface plasmon resonance (SPR) single cycle kinetics in which the antibody was immobilized by amide coupling to the sensor chip surface and various dilutions of compound (40, 20, 10, 5, 2.5 nM) were flowed across the sensor. The association (kₒₙ) and dissociation (k_{off}) rates were determined by fitting a 1:1 binding model to the data to arrive at affinity constants (K_{d}) determined for each compound (Table 2D). An exemplary sensorgram of the direct binding kinetics of JBZ-4 to fentanyl is shown in FIG. 8A and to carfentanil is shown in FIG. 8B. Affinities for fentanyl analogues were typically in the picomolar (pM) range.

TABLE 2D
Opioid Binding Kinetics of JBZ-4

| Compound | kₒₙ (M⁻¹ s⁻¹) | k_{off} (s⁻¹) | K_{d} (M) |
|---|---|---|---|
| Carfentanil | 3.39E+06 | 2.43E−04 | 7.18E−11 |
| Fentanyl | 9.46E+06 | 2.07E−03 | 2.19E−10 |
| 3-Methylfentanyl | 1.41E+07 | 9.72E−02 | 6.92E−09 |
| Acetylfentanyl | 1.10E+07 | 2.13E−03 | 1.94E−10 |
| α-Methylfentanyl | 6.50E+06 | 4.79E−03 | 7.37E−10 |
| Butyrylfentanyl | 1.47E+07 | 2.10E−03 | 1.43E−10 |
| p-Tolylfentanyl | 1.00E+07 | 1.73E−03 | 1.73E−10 |

Binding selectivity was also determined by competitive SPR assay, in which a fentanyl-BSA conjugate was immobilized to the sensor surface and JBZ-4 antibody mixed with various compounds was flowed over the chip surface. As shown in Table 2E, 16 nM of fentanyl or carfentanil completely inhibited sensor ligand binding while >1000× greater concentrations of non-fentanyl molecules resulted in very little compound binding. Binding selectivity was typically greater than 10⁴-fold for each molecule, although an exact value could not be determined due to highly insufficient antibody binding to non-fentanyl molecules.

TABLE 2E
Opioid Binding Selectivity of JBZ-4

| | | | | | Average | Compound | |
|---|---|---|---|---|---|---|---|
| | | Replicate | Replicate | Replicate | Response | Binding | Fold- |
| Compound | Concentration | 1 | 2 | 3 | (RU) | (%) | Selectivity |
| Naloxone | 30 μM | 83.33 | 69.07 | 67.73 | 73.38 | 4.7 | >10⁵ |
| Naltrexone | 30 μM | 71.11 | 66.89 | 52.76 | 63.59 | 17.4 | >10⁴ |
| Buprenorphine | 21 μM | 65.91 | 62.88 | 60.49 | 63.09 | 18.0 | >10⁴ |
| Norbuprenorphine | 24 μM | 64.04 | 53.15 | 57.97 | 58.39 | 24.1 | >10⁴ |
| Oxycodone | 20 μM | 60.09 | 59.65 | 54.98 | 58.24 | 24.3 | >10⁴ |
| Hydrocodone | 20 μM | 60.4 | 57.84 | 54.87 | 57.70 | 25.0 | >10⁴ |
| Alfentanil | 20 μM | 60.08 | 57.15 | 54.95 | 57.39 | 25.4 | >10⁴ |
| Remifentanil | 2 μM | 38.67 | 38.67 | 38.82 | 38.72 | 49.7 | >10³ |
| 6-Acetylmorphine | 2 μM | 64.52 | 61.08 | 59.22 | 61.61 | 20.0 | >10⁴ |
| Fentanyl | 15.6 nM | 4.25 | 3.25 | 3.58 | 3.69 | 95.2 | 2-3 |
| Carfentanil | 15.6 nM | 0.46 | −7.93 | −3.16 | −3.54 | 104.6 | 1 |
| No compound | 0 | 81.4 | 75.18 | 74.33 | 76.97 | 0 | N/A |
| control | | | | | | | |

### Example 3

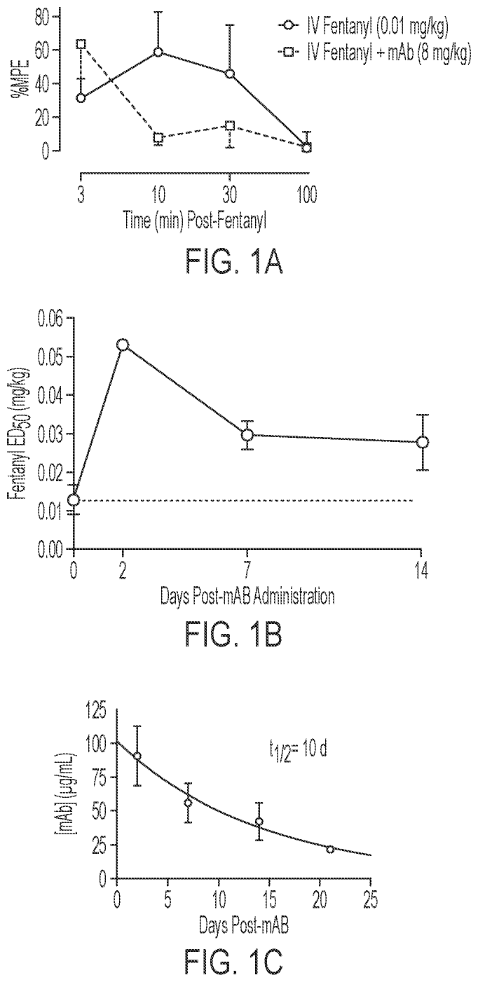

Rhesus monkey antinociception experiments were performed as previously described: Tenney et al, Neuropharmacology. 2019; 158:107730. Monkeys were administered IV fentanyl at a dose of 0.1-0.18 mg/kg followed by an IV injection of JBZ-1 or comparative mAb at 8 mg/kg. The effects of the antibody on antinociception were followed for the first 100 min after fentanyl injection and also at 2, 7 and 14 days. Blood samples collected from the monkeys were analyzed by ELISA against a fentanyl-BSA capture antigen using the corresponding antibodies for generating standard curves. The effects on tail withdrawal antinociception were observed and shown in FIG. 1A. Cumulative dose fentanyl ED^{50S} were determined in the same monkeys at three time points post mAb infusion, as shown in FIG. 1B. Blood samples were taken from the monkeys and analyzed by ELISA to determine antibody concentrations at the indicated time points shown in FIG. 1C.

### Example 4

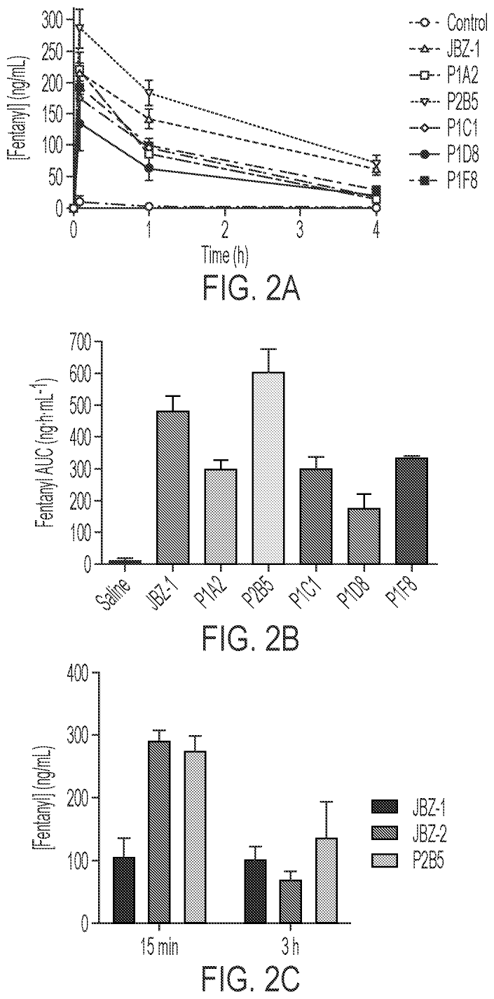

Female Swiss Webster mice (n=4 per group) were intravenously administered via tail vein injection 30 mg/kg mAb 48 h prior to an IV 0.1 mg/kg fentanyl citrate tail fentanyl tail vein injection. Blood samples were pulled at the indicated time points post-fentanyl and prepped as follows: frozen mouse blood samples were thawed on ice and 60 μL blood was pipetted into a new tube along with 8 μL of 50 ng/mL fentanyl-d5 in MeOH. After vortexing, 120 uL of 50 mM K2003 solution and 420 μL of 7:3 hexane/ethyl acetate were added. The samples were vortexed for 15 seconds and centrifuged at 3000 rpm for 5 min. The top solvent layers were pipetted into new tubes and evaporated by Genevac for 1 h. The resulting residues were dissolved in 68 μL MeOH and analyzed on an LC/MS/MS instrument. Standards were prepared in the same manner by extracting blood spiked with fentanyl ranging in concentration from 5000 ng/mL to 1.6 ng/mL. The limit of detection was determined to be around 1.6 ng/mL. The results of the time course experiment of the blood fentanyl levels in the mice are shown in FIG. 2A. FIG. 2B shows fentanyl area under curve values derived from panel FIG. 2A data. Fentanyl blood levels in the mice treated with JBZ-1, JBZ-2, and P2B5 at 15 min and 3 h, shown in FIG. 2C.

### Example 5

SCID mice (n=5-8 per group) were tested for cumulative drug response in primarily supraspinal (hot plate) and spinal (tail flick) behavioral tests 24 h after IV injection of a 25 mg/kg JBZ-1 dose. In the hot plate test, the mouse was observed in an acrylic cylinder (14 cm diameter×22 cm) on a 55° C. surface. The latency to perform one of the following nociceptive responses: licking of hind paw, shaking/withdrawal of hind paw or jumping, was timed with a 35 s cutoff to prevent tissue damage. The tail immersion test was performed using an ITC Life Science Tail Flick Analgesia Meter to measure time of tail withdrawal from a heated light beam (45% active intensity) with a cutoff of 10 s to prevent tissue damage. Since tail flick is a reflexive behavior, hot plate was always performed first. Immediately following both antinociceptive assays, fentanyl or carfentanil was injected intraperitoneally. The fentanyl doses tested were 0.2, 0.4, 0.6, and 0.8 mg/kg and the carfentanil doses tested were 0.01, 0.02, 0.04, and 0.08 mg/kg to generate a full dose-response curve. For the control, an extra set of (n=4) mice received smaller doses (0.05, 0.1, 0.15, 0.2 mg/kg fentanyl and 0.005, 0.01, 0.015 and mg/kg for carfentanil) to more accurately assess the ED₅₀. Testing for all animals was repeated in fifteen minute intervals, following each injection with increase cumulative dosing until full antinociception (cut off times surpassed) was observed in both assays. Percent maximum possible effect (% MPE) was calculated from time by the equation:

$$MPE = \frac{( test - b a s e l i n e )}{( cutoff - ba s e l ine )} \times 10$$

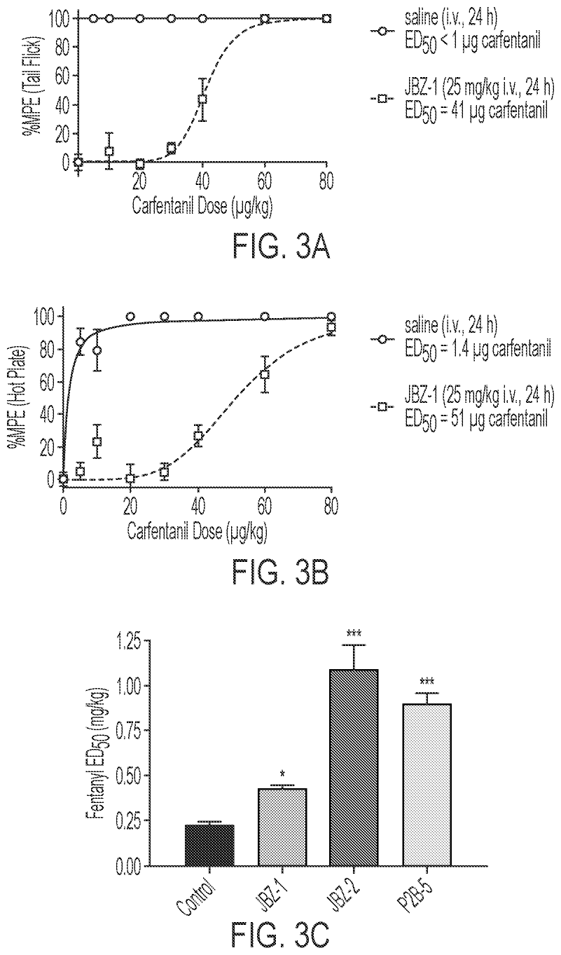

The resulting % MPE versus log(dose) curve was fit using a log (agonist) vs. normalized response non-linear regression in GraphPad PRISM 6. The ED₅₀ values are represented at the mean±SEM and were determined for each antinociception test and individual treatment groups. In vivo mAb-mediated antagonism of carfentanil and fentanyl-induced antinociception in mice. FIGS. 3A-3B show that mAb treatment 24 hours prior to drug challenge significantly shifts carfentanil dose-effect curves rightward in hot plate and flick tail antinociception tests. FIG. 3C shows a comparison of fentanyl ED₅₀ in mice treated with JBZ-1, JBZ-2, and P2B5 antibody. FIG. 3D is a comparison of the percentage of mice below the maximum possible effect cutoff in the hot plate antinociception assay when treated with JBZ-1, JBZ-2, and JBZ-3.

### Example 6

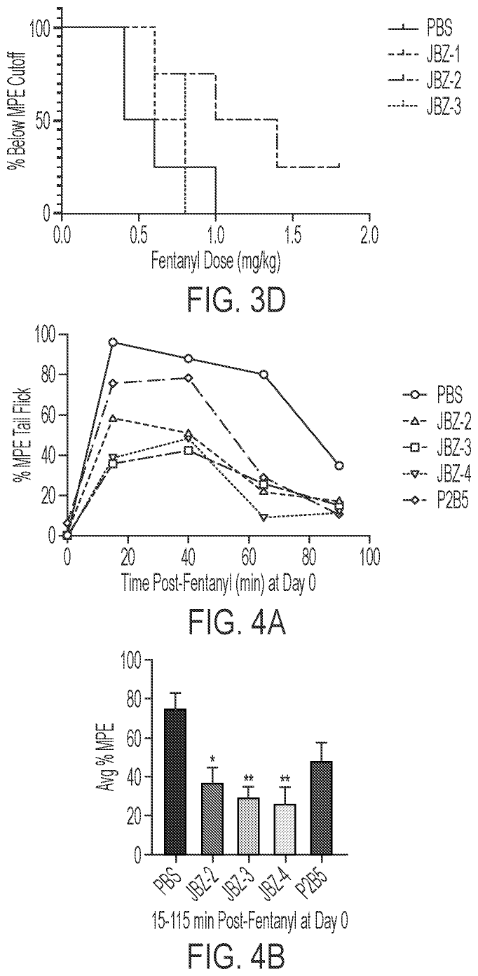

JBZ-2, JBZ-3, JBZ-4, and P2B5 antibodies were evaluated in fentanyl antinociception assay. The mAbs (45 mg/kg) were given IP to 4 Swiss Webster females and antinociception induced by 0.4 mg/kg IP fentanyl administered 4 h post-mAb. Measurements were taken over 90 minutes and the results are shown in FIG. 4A. Antinociception was followed for a 15-115 minute interval post-fentanyl and expressed as an average in FIG. 4B. Fentanyl antinociception was retested in the same mice at 2 days post-mAb and the results are shown in FIG. 4C.

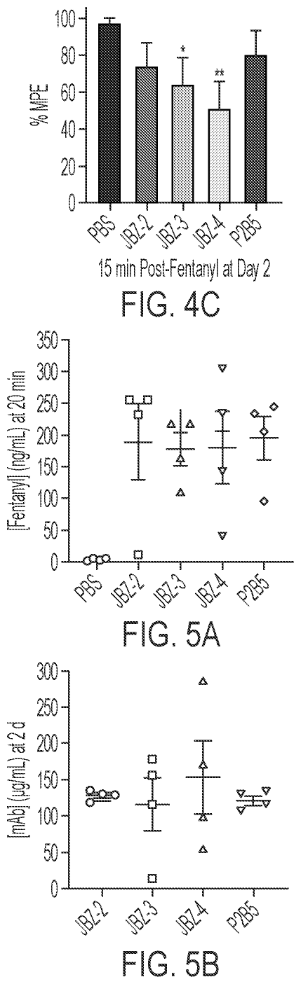

The same mice from the antinociception study were administered an IP fentanyl challenge (0.4 mg/kg), and blood samples were taken 20 min after drug injection. The results are shown in FIG. 5A. Samples were basified, combined with deuterated internal standard, extracted with organic solvent and analyzed by LC-MS/MS using a standard curve to interpolate unknown concentrations. Blood samples taken before drug administration at 2 d post-mAb administration were analyzed by ELISA. The results are shown in FIG. 5B.

### Example 7

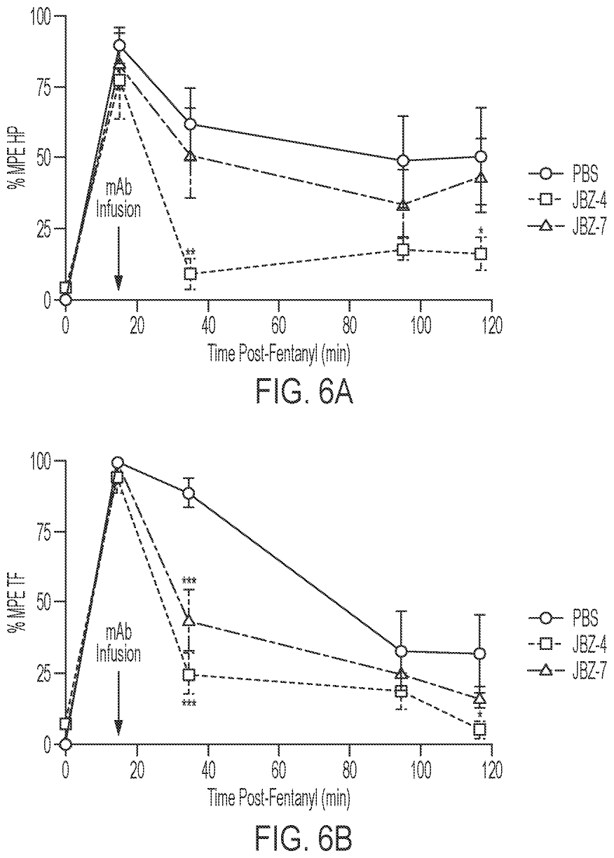

Rescue of fentanyl-induced antinociception by JBZ-4 and JBZ-7 infusion was tested. Following a 0.2 mg/kg IP dose of fentanyl, mice were tested in hot plate (FIG. 6A) and tail flick (FIG. 6B) antinociception assays at 15 min and immediately administered 30 mg/kg intravenous antibody after which testing resumed. JBZ-4 produced superior results when compared to JBZ-7.

### Example 8

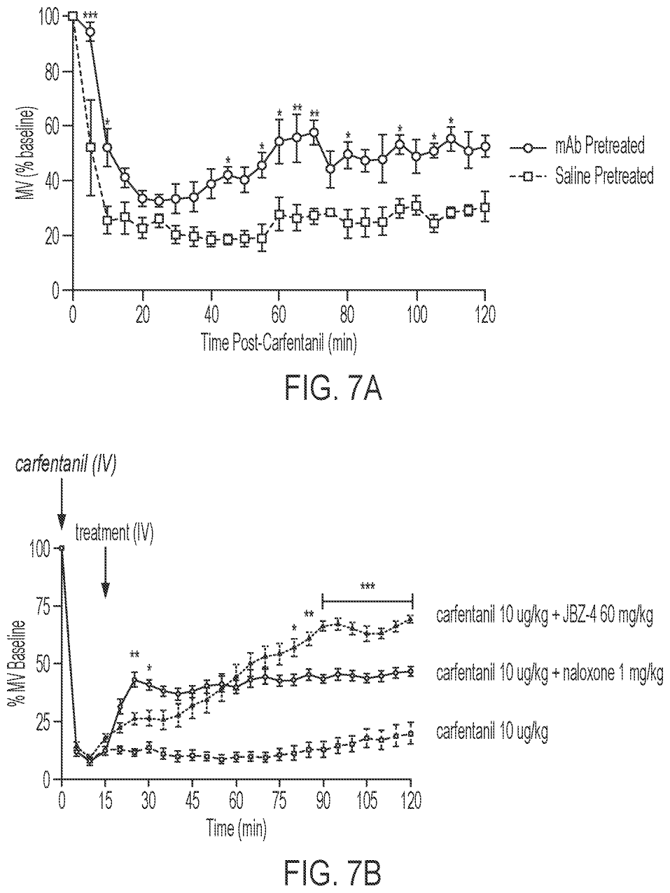

While the antinociception assay is a reliable indicator of opioid analgesia, respiration models are more translationally relevant to the fatal effects of opioids. The antibodies of the disclosure, e.g., JBZ-4, block and rescues carfentanil-induced respiratory depression. Mice pretreated with 30 mg/kg JBZ-4 were challenged 48 hours later with 30 μg/kg IV carfentanil and their respiration was observed by whole body plethysmography and expressed as minute volume (MV) normalized to baseline. The results are show in FIG. 7A.

Efficacy of JBZ-4 was assessed in a mouse model in which respiratory depression was induced by carfentanil followed by a rescue infusion of antibody. The experimental protocol consisted of first equilibrating mice (n=12 per group) in an EMKA whole body plethysmography apparatus for 20 min until stable respiration was observed to serve as a baseline reading. At time=0 min, carfentanil (10 μg/kg, IV) was administered and a steep drop in respiration was observed down to approximately 10% of baseline minute volume (MV). At time=15 min, JBZ-4 antibody (60 mg/kg, IV) or naloxone (1 mg/kg IV) was administered after which an immediate and steady increase of MV was observed, achieving statistical significance vs. the saline control group starting at 20 min. While naloxone showed a quicker onset of action due to its rapid distribution as a small-molecule, the antibody showed a more robust degree of reversal at later time points (FIG. 7B).

### Example 9

An x-ray crystal structure with about 1.8 Å resolution was elucidated of JBZ-4 antigen binding fragment (Fab) complexed with fentanyl. The structure reveals a 1:1 ratio of drug to Fab (or a 2:1 ratio of drug to full IgG), and an antigen binding region at the Fab tip formed between heavy and light chains. The structure also shows the fentanyl binding mode: the molecule is oriented with the phenethyl tail pointing ‘down’ into the paratope hydrophobic pocket, the propanoyl group appears to be pointing toward the solvent and the anilide ring appears to engage a shallow pocket adjacent to the deeper phenethyl pocket.

### Example 10

PK Study. A non-GLP single dose pharmacokinetic study of monoclonal antibody (JBZ-4) was conducted. Groups of nine adult CD male rats were administered a single intravenous (iv) bolus dose of monoclonal antibody JBZ-4 (10 mL/kg) at 10, 100, and 250 mg/kg. Blood samples for determination of serum concentrations of JBZ-4 were obtained from 3 rats/time point at 16 time points (0.083, 1, 4, 8, 24, 48, 72, 96, 144, 192, 240, 288, 336, 408, 504, and 672 hours post-dose). Blood samples were processed into serum and analyzed for JBZ-4 using an ELISA method. Group mean serum concentration-time profiles were used to estimate the following PK parameters in rats, using non-compartmental analysis (Phoenix WinNonlin, version 8.1, Certara, Princeton, NJ): total drug exposure defined as area under the serum concentration-time curve, mean β-phase half-life (t_{1/2β}), Cₘₐₓ, clearance (CL), and apparent volume of distribution of the terminal phase (Vz). The nominal dose administered for each treatment group was used for modeling.

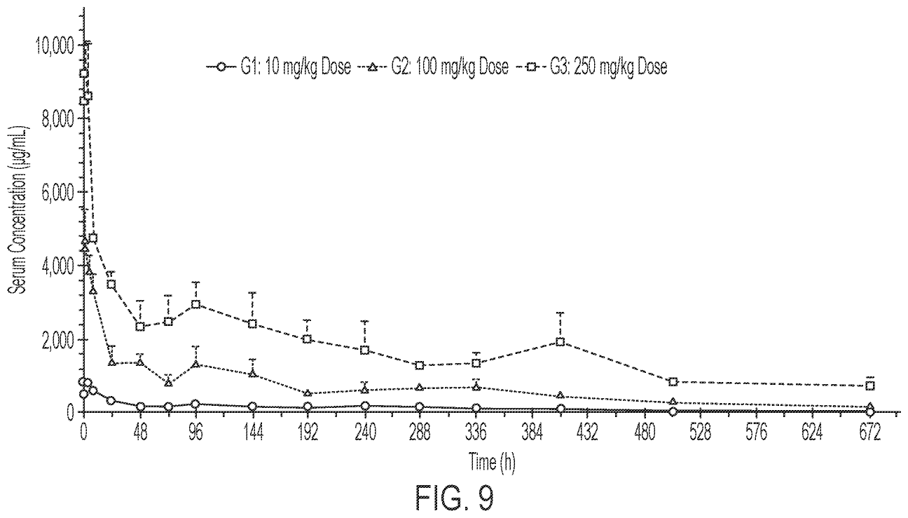

PK profiles of JBZ-4 in rats following a single i.v. bolus dose at 10, 25, and 250 mg/kg are shown in FIG. 9. Rats showed a bi-exponential serum concentration-time profile with a short distribution phase followed by a long elimination phase. Half-life, Cₘₐₓ, AUC, and volume of distribution (vz), increased proportionally with dose level are shown in Table 3. At 10 mg/kg, half-life was 102 hr, Cₘₐₓ was 805 pg/mL, volume of distribution was 0.0196 L/kg, and the clearance value (CL) was 0.134 mL/hr/kg. At 100 mg/kg, half-life was 171 hr, Cₘₐₓ was 4701 μg/mL, volume of distribution was 0.0485, and the clearance value was 0.197 mL/hr/kg. At 250 mg/kg, half-life was 308 hr, Cₘₐₓ was 9228 μg/mL, volume of distribution was 0.0766 L/kg, and the clearance value was 0.173 mL/hr/kg. Bi-phasic distribution of JBZ-4 with a long elimination half-life is characteristic of IgG monoclonal antibodies.

TABLE 3
PK in rats following a single intravenous bolus dose of JBZ-4

| JBZ-4 | Half- | Cₘₐₓ | AUCₗₐₛₜ | AUC_{inf} | | CL |
|---|---|---|---|---|---|---|
| Dose | life | (μg/ | (hr * | (hr * | v_{Z} | (mL/ |
| (mg/kg) | (hr) | mL) | [μg/mL]) | [μg/mL]) | (L/kg) | hr/kg) |
| 10 | 102 | 805 | 73301 | 74825 | 0.0196 | 0.134 |
| 100 | 171 | 4701 | 463889 | 508222 | 0.0485 | 0.197 |
| 250 | 308 | 9228 | 1132976 | 1448827 | 0.07660 | 0.173 |

### Example 11

A monkey plethysmography study will be conducted similar to the procedures described in Kishioka et al, Buprenorphine and methoclocinnamox: agonist and antagonist effects on respiratory function in rhesus monkeys,” European Journal of Pharmacology, 391:289-297 (2000), except that JBZ-4 will be used instead of buprenorphine or methoclocinnamox, and fentanyl or fentanyl analogs will be used instead of morphine or heroin. The study will evaluate the ability of JBZ-4 to antagonize the respiratory suppressant effects of fentanyl. Briefly, monkeys will be pretreated with intravenous or intramuscular injections of 1 mg/kg to 40 mg/kg of JBZ-4. During testing sessions, intramuscular cumulative doses of 0.0001 mg/kg to 0.01 mg/kg fentanyl will be administered to monkeys, immediately followed by exposure to air for 14 minutes then exposure to 5% carbon dioxide for 6 minutes. This cycle will be conducted 3-5 times. The study will generate an opioid dose response curve that will be shifted rightward upon treatment with JBZ-4. Dose response curves generated with non-fentanyl opioids such as oxycodone should be unaffected by JBZ-4 treatment.

The section headings used herein are for organizational purposes only and are not to be construed as limiting the subject matter described. All documents, or portions of documents, cited in the application are hereby expressly incorporated by reference in their entirety for any purpose.

While various embodiments and aspects are shown and described herein, it will be obvious to those skilled in the art that such embodiments and aspects are provided by way of example only. Numerous variations, changes, and substitutions will now occur to those skilled in the art. Various alternatives to the embodiments and aspects described herein may be used.

Informal Sequence Listing

The position of CDRs and FRs in the sequences described herein are defined by the Kabat numbering system.

SEQ ID NO: 1 = CDR-L1
SGDTLPKRSGY
SEQ ID NO: 2 = CDR-L2
KDTERPS
SEQ ID NO: 3 = CDR-L3
QSADSSFTYPS
SEQ ID NO: 4 = CDR-H1
SRNWWS
SEQ ID NO: 5 = CDR-H2
EVYHTGITNYNPSLKS
SEQ ID NO: 6 = CDR-H3
EVVGPTTGYFDL
SEQ ID NO: 7 = LFR1
SYELTQPPSVSVSPGQTARITC
SEQ ID NO: 8 = LFR2
WYQQKPDQAPLLVIN
SEQ ID NO: 9 = LFR3
GIPERFSGSKSGTTVTLTISGVQAEDEADYYC
SEQ ID NO: 10 = LFR4
FGGGTKLTVL
SEQ ID NO: 11 = HFR1
QVQLQESGPGLVKPSGTLSLTCTVSGGFIS
SEQ ID NO: 12 = HFR2
WVRQPPGKGLEWIG
SEQ ID NO: 13 = HFR3
RVTISVDKSKNQFSLKLSSVTAADTAVYYCAR
SEQ ID NO: 14 = HFR4
WGRGTLVTISS
SEQ ID NO: 15 = CDR-H1
GGFISSRN
SEQ ID NO: 16 = CDR-H2
YHTGI
SEQ ID NO: 17 = HFR1
QVQLQESGPGLVKPSGTLSLTCTVS
SEQ ID NO: 18 = HFR2
WWSWVRQPPGKGLEWIGEV
SEQ ID NO: 19 = HFR3
TNYNPSLKSRVTISVDKSKNQFSLKLSSVTAADTAVYYCAR
SEQ ID NO: 20 = Light Chain Variable Region.
SYELTQPPSVSVSPGQTARITCSGDTLPKRSGYWYQQKPDQAPLLVINKDTERPSGIPERFSGS
KSGTTVTLTISGVQAEDEADYYCQSADSSFTYPSFGGGTKLTVL
SEQ ID NO: 21 = Light Chain Constant Region
GQPKAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECS
SEQ ID NO: 22 = Light Chain Amino Acid Sequence
SYELTQPPSVSVSPGQTARITCSGDTLPKRSGYWYQQKPDQAPLLVINKDTERPSGIPERFSGS
KSGTTVTLTISGVQAEDEADYYCQSADSSFTYPSFGGGTKLTVLGQPKAAPSVTLFPPSSEELQ
ANKATLVCLISDFYPGAVTVAWKADSSPVKAGVETTTPSKQSNNKYAASSYLSLTPEQWKSHRS
YSCQVTHEGSTVEKTVAPTECS
SEQ ID NO: 23: Heavy Chain Variable Region.
QVQLQESGPGLVKPSGTLSLTCTVSGGFISSRNWWSWVROPPGKGLEWIGEVYHTGITNYNPSL
KSRVTISVDKSKNQFSLKLSSVTAADTAVYYCAREVVGPTTGYFDLWGRGTLVTISS
SEQ ID NO: 24 = Heavy Chain Constant Region
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHNHYT
QKSLSLSPGK
SEQ ID NO: 25 = Heavy Chain Amino Acid Sequence
QVQLQESGPGLVKPSGTLSLTCTVSGGFISSRNWWSWVRQPPGKGLEWIGEVYHTGITNYNPSL
KSRVTISVDKSKNQFSLKLSSVTAADTAVYYCAREVVGPTTGYFDLWGRGTLVTISSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLS
PGK
SEQ ID NO: 26
JBZ-2 Light Chain Nucleic Acid Sequence
TCCTATGAGCTGACCCAGCCACCCTCCGTGTCTGTGAGCCCAGGCCAGACCGCCAGGATCACAT
GTTCCGGCGATACACTGCCCAAGCGGAGCGGCTACTGGTATCAGCAGAAGCCAGACCAGGCCCC
CCTGCTGGTCATCAACAAGGATACCGAGAGGCCTTCTGGCATCCCAGAGCGGTTCAGCGGCAGC
AAGTCTGGCACCACAGTGACCCTGACAATCAGCGGAGTGCAGGCAGAGGACGAGGCAGATTACT
ATTGCCAGTCCGCCGACAGCTCCTTCACCTACCCAAGCTTTGGCGGCGGCACCAAGCTGACAGT
GCTGGGTCAGCCCAAGGCTGCCCCCTCGGTCACTCTGTTCCCGCCCTCGAGTGAGGAGCTTCAA
GCCAACAAGGCCACACTGGTGTGTCTCATAAGTGACTTCTACCCGGGAGCCGTGACAGTGGCCT
GGAAGGCAGATAGCAGCCCCGTCAAGGCGGGAGTGGAGACCACCACACCCTCCAAACAAAGCAA
CAACAAGTACGCGGCCAGCAGCTACCTGAGCCTGACGCCTGAGCAGTGGAAGTCCCACAGAAGC
TACAGCTGCCAGGTCACGCATGAAGGGAGCACCGTGGAGAAGACAGTGGCCCCTACAGAATGTT
CA
SEQ ID NO: 27
JBZ-2 Light Chain Nucleic Acid Sequence
TCCTATGAGCTGACCCAGCCACCCTCCGTGTCTGTGAGCCCAGGCCAGACCGCCAGGATCACAT
GTTCCGGCGATACACTGCCCAAGCGGAGCGGCTACTGGTATCAGCAGAAGCCAGACCAGGCCCC
CCTGCTGGTCATCAACAAGGATACCGAGAGGCCTTCTGGCATCCCAGAGCGGTTCAGCGGCAGC
AAATCTGGCACCACAGTGACCCTGACAATCAGCGGAGTGCAGGCAGAGGACGAGGCAGATTACT
ATTGCCAGTCCGCCGACAGCTCCTTCACCTACCCAAGCTTTGGCGGCGGCACCAAGCTGACAGT
GCTGGGTCAGCCCAAGGCTGCCCCCTCGGTCACTCTGTTCCCGCCCTCGAGTGAGGAGCTTCAA
GCCAACAAGGCCACACTGGTGTGTCTCATAAGTGACTTCTACCCGGGAGCCGTGACAGTGGCCT
GGAAGGCAGATAGCAGCCCCGTCAAGGCGGGAGTGGAGACCACCACACCCTCCAAACAAAGCAA
CAACAAGTACGCGGCCAGCAGCTACCTGAGCCTGACGCCTGAGCAGTGGAAGTCCCACAGAAGC
TACAGCTGCCAGGTCACGCATGAAGGGAGCACCGTGGAGAAGACAGTGGCCCCTACAGAATGTT
CA
SEQ ID NO: 28
JBZ-2 Heavy Chain Nucleic Acid Sequence
CAGGTGCAGCTGCAGGAGAGCGGACCTGGACTGGTGAAGCCATCCGGCACCCTGTCTCTGACCT
GCACAGTGTCTGGCGGCTTCATCAGCTCCCGGAACTGGTGGAGCTGGGTGAGACAGCCACCTGG
CAAGGGCCTGGAGTGGATCGGCGAGGTGTACCACACCGGCATCACAAACTATAACCCCTCCCTG
AAGTCTCGGGTGACCATCAGCGTGGACAAGTCCAAGAATCAGTTCAGCCTGAAGCTGTCTAGCG
TGACCGCCGCCGATACAGCCGTGTACTATTGCGCCAGGGAGGTGGTGGGACCTACCACAGGCTA
CTTTGACCTGTGGGGAAGGGGCACCCTGGTGACAATCTCCTCTGCGTCGACCAAGGGCCCATCG
GTCTTCCCCCTGGCACCCTCCTCCAAGAGCACCTCTGGGGGCACAGCGGCCCTGGGCTGCCTGG
TCAAGGACTACTTCCCCGAACCTGTGACGGTCTCGTGGAACTCAGGCGCCCTGACCAGCGGCGT
GCACACCTTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAGCAACACCA
AGGTGGACAAGAAGGTTGAGCCCAAATCTTGTGACAAAACTCACACATGCCCACCGTGCCCAGC
ACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCA
GTACAACAGCACGTACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGATGAGCTGAC
CAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTT
CTCATGCTCCGTGATGCATGAGGCTCTGCACAACCACTACACGCAGAAGAGCCTCTCCCTGTCC
CCGGGTAAA
SEQ ID NO: 29 Custom CDR-L3
xQSAxFTYPx
SEQ ID NO: 30 Custom CDR-L3
xQSADSSFTYPSX
SEQ ID NO: 31 Custom CDR-H1
GGFISSRNW
SEQ ID NO: 32 Custom CDR-H2
xWxExIxNx
SEQ ID NO: 33 Custom CDR-H2
xWxEXITNx
SEQ ID NO: 34 Custom CDR-H3
xExVxGYFx
SEQ ID NO: 35 Custom CDR-H3
xExVGPxTGYFx
SEQ ID NO: 36 = LFR3 JBZ-2
GIPERFSGSSSGTTVTLTISGVQAEDEADYYC
SEQ ID NO: 37 = Light Chain Variable Region. JBZ-2
SYELTQPPSVSVSPGQTARITCSGDTLPKRSGYWYQQKPDQAPLLVINKDTERPSGIPERFSGS
SSGTTVTLTISGVQAEDEADYYCQSADSSFTYPSFGGGTKLTVL
SEQ ID NO: 38 = Light Chain Amino Acid Sequence. JBZ-2
SYELTQPPSVSVSPGQTARITCSGDTLPKRSGYWYQQKPDQAPLLVINKDTERPSGIPERFSGS
SSGTTVTLTISGVQAEDEADYYCQSADSSFTYPSFGGGTKLTVLGQPKAAPSVTLFPPSSEELQ
ANKATLVCLISDFYPGAVTVAWKADSSPVKAGVETTTPSKQSNNKYAASSYLSLTPEQWKSHRS
YSCQVTHEGSTVEKTVAPTECS
SEQ ID NO: 39 = LFR3 JBZ-6
GIPERFSGSNSGTTVTLTISGVQAEDEADYYC
SEQ ID NO: 40 = Light Chain Variable Region. JBZ-6
SYELTQPPSVSVSPGQTARITCSGDTLPKRSGYWYQQKPDQAPLLVINKDTERPSGIPERFSGS
NSGTTVTLTISGVQAEDEADYYCQSADSSFTYPSFGGGTKLTVL
SEQ ID NO: 41 = Light Chain Amino Acid Sequence JBZ-6
SYELTQPPSVSVSPGQTARITCSGDTLPKRSGYWYQQKPDQAPLLVINKDTERPSGIPERFSGS
NSGTTVTLTISGVQAEDEADYYCQSADSSFTYPSFGGGTKLTVLGQPKAAPSVTLEPPSSEELQ
ANKATLVCLISDFYPGAVTVAWKADSSPVKAGVETTTPSKQSNNKYAASSYLSLTPEQWKSHRS
YSCQVTHEGSTVEKTVAPTECS
SEQ ID NO: 42 = LFR3 JBZ-5
GIPERFSGSTSGTTVTLTISGVQAEDEADYYC
SEQ ID NO: 43 = Light Chain Variable Region. JBZ-5
SYELTQPPSVSVSPGQTARITCSGDTLPKRSGYWYQQKPDQAPLLVINKDTERPSGIPERFSGS
TSGTTVTLTISGVQAEDEADYYCQSADSSFTYPSFGGGTKLTVL
SEQ ID NO: 44 = Light Chain Amino Acid Sequence JBZ-5
SYELTQPPSVSVSPGQTARITCSGDTLPKRSGYWYQQKPDQAPLLVINKDTERPSGIPERFSGS
TSGTTVTLTISGVQAEDEADYYCQSADSSFTYPSFGGGTKLTVLGQPKAAPSVTLEPPSSEELQ
ANKATLVCLISDFYPGAVTVAWKADSSPVKAGVETTTPSKOSNNKYAASSYLSLTPEQWKSHRS
YSCQVTHEGSTVEKTVAPTECS
SEQ ID NO: 45
JBZ-1 Light Chain Amino Acid Sequence:
SYELTQPPSVSVSPGQTARITCSGDTLPKRSGYWYQQKPDQAPLLVINKDTERPSGIPERFSGS
SSGTTVTLTISGVQAEDEADYYCOSADSSFTYPMFGGGTKLTVLGOPKAAPSVTLFPPSSEELQ
ANKATLVCLISDFYPGAVTVAWKADSSPVKAGVETTTPSKQSNNKYAASSYLSLTPEQWKSHRS
YSCQVTHEGSTVEKTVAPTECS
SEQ ID NO: 46
JBZ-1 Heavy Chain Amino Acid Sequence:
QVQLQESGPGLVKPSGTLSLTCTVSGGFISSRNWWSWVROPPGKGLEWIGEVYHTGITNYIPSL
KSRVTISVDKSKNQFSLKLSSVTAADTAVYYCAREVVGPTTGYFDLWGRGTLVTISSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLS
PGK
SEQ ID NO: 47
JBZ-1 Light Chain Nucleic Acid Sequence:
TCCTATGAGCTGACCCAGCCACCCTCCGTGTCTGTGAGCCCAGGCCAGACCGCCAGGATCACAT
GTTCCGGCGATACACTGCCCAAGCGGAGCGGCTACTGGTATCAGCAGAAGCCAGACCAGGCCCC
CCTGCTGGTCATCAACAAGGATACCGAGAGGCCTTCTGGCATCCCAGAGCGGTTCAGCGGCAGC
TCCTCTGGCACCACAGTGACCCTGACAATCAGCGGAGTGCAGGCAGAGGACGAGGCAGATTACT
ATTGCCAGTCCGCCGACAGCTCCTTCACCTACCCAATGTTTGGCGGCGGCACCAAGCTGACAGT
GCTGGGTCAGCCCAAGGCTGCCCCCTCGGTCACTCTGTTCCCGCCCTCGAGTGAGGAGCTTCAA
GCCAACAAGGCCACACTGGTGTGTCTCATAAGTGACTTCTACCCGGGAGCCGTGACAGTGGCCT
GGAAGGCAGATAGCAGCCCCGTCAAGGCGGGAGTGGAGACCACCACACCCTCCAAACAAAGCAA
CAACAAGTACGCGGCCAGCAGCTACCTGAGCCTGACGCCTGAGCAGTGGAAGTCCCACAGAAGC
TACAGCTGCCAGGTCACGCATGAAGGGAGCACCGTGGAGAAGACAGTGGCCCCTACAGAATGTT
CA
SEQ ID NO: 48 JBZ-1 Heavy Chain Nucleic Acid Sequence:
CAGGTGCAGCTGCAGGAGAGCGGACCTGGACTGGTGAAGCCATCCGGCACCCTGTCTCTGACCT
GCACAGTGTCTGGCGGCTTCATCAGCTCCCGGAACTGGTGGAGCTGGGTGAGACAGCCACCTGG
CAAGGGCCTGGAGTGGATCGGCGAGGTGTACCACACCGGCATCACAAACTATATCCCCTCCCTG
AAGTCTCGGGTGACCATCAGCGTGGACAAGTCCAAGAATCAGTTCAGCCTGAAGCTGTCTAGCG
TGACCGCCGCCGATACAGCCGTGTACTATTGCGCCAGGGAGGTGGTGGGACCTACCACAGGCTA
CTTTGACCTGTGGGGAAGGGGCACCCTGGTGACAATCTCCTCTGCGTCGACCAAGGGCCCATCG
GTCTTCCCCCTGGCACCCTCCTCCAAGAGCACCTCTGGGGGCACAGCGGCCCTGGGCTGCCTGG
TCAAGGACTACTTCCCCGAACCTGTGACGGTCTCGTGGAACTCAGGCGCCCTGACCAGCGGCGT
GCACACCTTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAGCAACACCA
AGGTGGACAAGAAGGTTGAGCCCAAATCTTGTGACAAAACTCACACATGCCCACCGTGCCCAGC
ACCTGAACTCCTGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCA
GTACAACAGCACGTACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGATGAGCTGAC
CAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTT
CTCATGCTCCGTGATGCATGAGGCTCTGCACAACCACTACACGCAGAAGAGCCTCTCCCTGTCC
CCGGGTAAA

What is claimed is:

1. An antibody comprising:
(i) a light chain variable region which comprises a CDR L1 having an amino acid sequence with at least 95% sequence identity to SEQ ID NO:1, a CDR L2 having an amino acid sequence with at least 95% sequence identity to SEQ ID NO:2, and a CDR L3 having an amino acid sequence with at least 95% sequence identity to SEQ ID NO:3; and
(ii) a heavy chain variable region which comprises: a CDR H1 having an amino acid sequence with at least 95% sequence identity to SEQ ID NO:15, a CDR H2 having an amino acid sequence with at least 95% sequence identity to SEQ ID NO:16, and a CDR H3 having an amino acid sequence with at least 95% sequence identity to SEQ ID NO:6.

2. The antibody of claim 1, wherein:
(i) the light chain variable region comprises CDR L1 as set forth in SEQ ID NO:1, CDR L2 as set forth in SEQ ID NO:2, and CDR L3 as set forth in SEQ ID NO:3; and
(ii) the heavy chain variable region comprises CDR H1 as set forth in SEQ ID NO:15, CDR H2 as set forth in SEQ ID NO:16, and CDR H3 as set forth in SEQ ID NO:6.

3. The antibody of claim 1 or 2, further comprising a light chain framework region which comprises a LFR1 having an amino acid sequence with at least 95% sequence identity to SEQ ID NO:7, a LFR2 having an amino acid sequence with at least 95% sequence identity to SEQ ID NO:8, a LFR3 having an amino acid sequence with at least 95% sequence identity to SEQ ID NO:9, and a LFR4 having an amino acid sequence with at least 95% sequence identity to SEQ ID NO:10.

4. The antibody of claim 3, wherein the light chain framework region comprises LFR1 as set forth in SEQ ID NO:7, LFR2 as set forth in SEQ ID NO:8, LFR3 as set forth in SEQ ID NO:9, and LFR4 as set forth in SEQ ID NO:10.

5. The antibody of any one of claims 1 to 4, further comprising a heavy chain framework region which comprises a HFR1 having an amino acid sequence with at least 95% sequence identity to SEQ ID NO:17; a HFR2 having an amino acid sequence with at least 95% sequence identity to SEQ ID NO:18, a HFR3 having an amino acid sequence with at least 95% sequence identity to SEQ ID NO:19, and a HFR4 having an amino acid sequence with at least 95% sequence identity to SEQ ID NO:14.

6. The antibody of claim 5, wherein the heavy chain framework region comprises HFR1 as set forth in SEQ ID NO:17; HFR2 as set forth in SEQ ID NO:18, HFR3 as set forth in SEQ ID NO:19, and HFR4 as set forth in SEQ ID NO:14.

7. An antibody comprising:
(i) a light chain variable region which comprises a CDR L1 having an amino acid sequence with at least 95% sequence identity to SEQ ID NO:1, a CDR L2 having an amino acid sequence with at least 95% sequence identity to SEQ ID NO:2, and a CDR L3 having an amino acid sequence with at least 95% sequence identity to SEQ ID NO:3; and
(ii) a heavy chain variable region which comprises a CDR H1 having an amino acid sequence with at least 95% sequence identity to SEQ ID NO:4, a CDR H2 having an amino acid sequence with at least 95% sequence identity to SEQ ID NO:5, and a CDR H3 having an amino acid sequence with at least 95% sequence identity to SEQ ID NO:6.

8. The antibody of claim 7, wherein:
(i) the light chain variable region which comprises CDR L1 as set forth in SEQ ID NO:1, CDR L2 as set forth in SEQ ID NO:2, and CDR L3 as set forth in SEQ ID NO:3; and
(ii) the heavy chain variable region which comprises CDR H1 as set forth in SEQ ID NO:4, CDR H2 as set forth in SEQ ID NO:5, and CDR H3 as set forth in SEQ ID NO:6.

9. The antibody of claim 7 or 8, further comprising a light chain framework region which comprises a LFR1 having an amino acid sequence with at least 95% sequence identity to SEQ ID NO:7, a LFR2 having an amino acid sequence with at least 95% sequence identity to SEQ ID NO:8, a LFR3 having an amino acid sequence with at least 95% sequence identity to SEQ ID NO:9, and a LFR4 having an amino acid sequence with at least 95% sequence identity to SEQ ID NO:10.

10. The antibody of claim 9, wherein the light chain framework region comprises LFR1 as set forth in SEQ ID NO:7, LFR2 as set forth in SEQ ID NO:8, LFR3 as set forth in SEQ ID NO:9, and LFR4 as set forth in SEQ ID NO:10.

11. The antibody of any one of claims 7 to 10, further comprising a heavy chain framework region which comprises a HFR1 having an amino acid sequence with at least 95% sequence identity to SEQ ID NO:11; a HFR2 having an amino acid sequence with at least 95% sequence identity to SEQ ID NO:12, a HFR3 having an amino acid sequence with at least 95% sequence identity to SEQ ID NO:13, and a HFR4 having an amino acid sequence with at least 95% sequence identity to SEQ ID NO:14.

12. The antibody of claim 11, wherein the heavy chain framework region comprises HFR1 as set forth in SEQ ID NO:11; HFR2 as set forth in SEQ ID NO:12, HFR3 as set forth in SEQ ID NO:13, and HFR4 as set forth in SEQ ID NO:14.

13. An antibody comprising a light chain variable region having at least 85% sequence identity to SEQ ID NO:20 and a heavy chain variable region having at least 85% sequence identity to SEQ ID NO:23.

14. The antibody of claim 13, wherein the light chain variable region comprises SEQ ID NO:20 and the heavy chain variable region comprises SEQ ID NO:23.

15. The antibody of any one of claims 1 to 14, further comprising a light chain constant region having an amino acid sequence with at least 85% identity to SEQ ID NO:21.

16. The antibody of claim 15, wherein the light chain constant region comprises SEQ ID NO:21.

17. The antibody of any one of claims 1 to 16, further comprising a heavy chain constant region with at least 85% identity to SEQ ID NO:24.

18. The antibody of claim 17, wherein the heavy chain constant region comprises SEQ ID NO:24.

19. The antibody of any one of claims 1 to 18, wherein the antibody is an IgG antibody.

20. The antibody of claim 19, wherein the IgG antibody is an IgG1 antibody.

21. The antibody of any one of claims 1 to 20, wherein the antibody is a Fab′ fragment or a F(ab)′₂ fragment.

22. The antibody of any one of claims 1 to 19, wherein the antibody is a single chain variable fragment.

23. The antibody of any one of claims 1 to 22, wherein the antibody is a humanized monoclonal antibody.

24. A pharmaceutical composition comprising the antibody of any one of claims 1 to 23 and a pharmaceutically acceptable excipient.

25. A method of treating or preventing an opioid overdose in a patient in need thereof, the method comprising administering to the patient an effective amount of the antibody of any one of claims 1 to 23 or the pharmaceutical composition of claim 24.

26. A method of treating opioid use disorder in a patient in need thereof, the method comprising administering to the patient an effective amount of the antibody of any one of claims 1 to 23 or the pharmaceutical composition of claim 24.

27. A method of treating or preventing opioid-induced respiratory depression in a patient in need thereof, the method comprising administering to the patient an effective amount of the antibody of any one of claims 1 to 23 or the pharmaceutical composition of claim 24.

28. An isolated nucleic acid encoding the antibody of any one of claims 1 to 23.

29. An isolated nucleic acid having a nucleic acid sequence with at least 85% sequence identity to SEQ ID NO:26, SEQ ID NO:27, or SEQ ID NO:28.

30. A vector comprising a nucleic acid encoding the antibody of any one of claims 1 to 23.

31. A complex comprising: (i) the antibody of any one of claims 1 to 23; and (ii) fentanyl, a fentanyl analogue, carfentanil, or a carfentanil analogue.


SEQUENCE LISTING (48 sequences)

SEQ ID NO: 1 - protein, 11 aa, Artificial Sequence
  Synthetic Construct
SGDTLPKRSGY

SEQ ID NO: 2 - protein, 7 aa, Artificial Sequence
  Synthetic Construct
KDTERPS

SEQ ID NO: 3 - protein, 11 aa, Artificial Sequence
  Synthetic Construct
QSADSSFTYPS

SEQ ID NO: 4 - protein, 6 aa, Artificial Sequence
  Synthetic Construct
SRNWWS

SEQ ID NO: 5 - protein, 16 aa, Artificial Sequence
  Synthetic Construct
EVYHTGITNYNPSLKS

SEQ ID NO: 6 - protein, 12 aa, Artificial Sequence
  Synthetic Construct
EVVGPTTGYFDL

SEQ ID NO: 7 - protein, 22 aa, Artificial Sequence
  Synthetic Construct
SYELTQPPSVSVSPGQTARITC

SEQ ID NO: 8 - protein, 15 aa, Artificial Sequence
  Synthetic Construct
WYQQKPDQAPLLVIN

SEQ ID NO: 9 - protein, 32 aa, Artificial Sequence
  Synthetic Construct
GIPERFSGSKSGTTVTLTISGVQAEDEADYYC

SEQ ID NO: 10 - protein, 10 aa, Artificial Sequence
  Synthetic Construct
FGGGTKLTVL

SEQ ID NO: 11 - protein, 30 aa, Artificial Sequence
  Synthetic Construct
QVQLQESGPGLVKPSGTLSLTCTVSGGFIS

SEQ ID NO: 12 - protein, 14 aa, Artificial Sequence
  Synthetic Construct
WVRQPPGKGLEWIG

SEQ ID NO: 13 - protein, 32 aa, Artificial Sequence
  Synthetic Construct
RVTISVDKSKNQFSLKLSSVTAADTAVYYCAR

SEQ ID NO: 14 - protein, 11 aa, Artificial Sequence
  Synthetic Construct
WGRGTLVTISS

SEQ ID NO: 15 - protein, 8 aa, Artificial Sequence
  Synthetic Construct
GGFISSRN

SEQ ID NO: 16 - protein, 5 aa, Artificial Sequence
  Synthetic Construct
YHTGI

SEQ ID NO: 17 - protein, 25 aa, Artificial Sequence
  Synthetic Construct
QVQLQESGPGLVKPSGTLSLTCTVS

SEQ ID NO: 18 - protein, 19 aa, Artificial Sequence
  Synthetic Construct
WWSWVRQPPGKGLEWIGEV

SEQ ID NO: 19 - protein, 41 aa, Artificial Sequence
  Synthetic Construct
TNYNPSLKSRVTISVDKSKNQFSLKLSSVTAADTAVYYCAR

SEQ ID NO: 20 - protein, 108 aa, Artificial Sequence
  Synthetic Construct
SYELTQPPSVSVSPGQTARITCSGDTLPKRSGYWYQQKPDQAPLLVINKDTERPSGIPERFSGSKSGTTVTLTISGVQAEDEADYYCQSADSSFTYPSFGGGTKLTVL

SEQ ID NO: 21 - protein, 106 aa, Artificial Sequence
  Synthetic Construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - protein, 214 aa, Artificial Sequence
  Synthetic Construct
SYELTQPPSVSVSPGQTARITCSGDTLPKRSGYWYQQKPDQAPLLVINKDTERPSGIPERFSGSKSGTTVTLTISGVQAEDEADYYCQSADSSFTYPSFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 121 aa, Artificial Sequence
  Synthetic Construct
QVQLQESGPGLVKPSGTLSLTCTVSGGFISSRNWWSWVRQPPGKGLEWIGEVYHTGITNYNPSLKSRVTISVDKSKNQFSLKLSSVTAADTAVYYCAREVVGPTTGYFDLWGRGTLVTISS

SEQ ID NO: 24 - protein, 330 aa, Artificial Sequence [sequence omitted]

SEQ ID NO: 25 - protein, 451 aa, Artificial Sequence
  Synthetic Construct
QVQLQESGPGLVKPSGTLSLTCTVSGGFISSRNWWSWVRQPPGKGLEWIGEVYHTGITNYNPSLKSRVTISVDKSKNQFSLKLSSVTAADTAVYYCAREVVGPTTGYFDLWGRGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 642 aa, Artificial Sequence
  Synthetic Construct
TCCTATGAGCTGACCCAGCCACCCTCCGTGTCTGTGAGCCCAGGCCAGACCGCCAGGATCACATGTTCCGGCGATACACTGCCCAAGCGGAGCGGCTACTGGTATCAGCAGAAGCCAGACCAGGCCCCCCTGCTGGTCATCAACAAGGATACCGAGAGGCCTTCTGGCATCCCAGAGCGGTTCAGCGGCAGCAAGTCTGGCACCACAGTGACCCTGACAATCAGCGGAGTGCAGGCAGAGGACGAGGCAGATTACTATTGCCAGTCCGCCGACAGCTCCTTCACCTACCCAAGCTTTGGCGGCGGCACCAAGCTGACAGTGCTGGGTCAGCCCAAGGCTGCCCCCTCGGTCACTCTGTTCCCGCCCTCGAGTGAGGAGCTTCAAGCCAACAAGGCCACACTGGTGTGTCTCATAAGTGACTTCTACCCGGGAGCCGTGACAGTGGCCTGGAAGGCAGATAGCAGCCCCGTCAAGGCGGGAGTGGAGACCACCACACCCTCCAAACAAAGCAACAACAAGTACGCGGCCAGCAGCTACCTGAGCCTGACGCCTGAGCAGTGGAAGTCCCACAGAAGCTACAGCTGCCAGGTCACGCATGAAGGGAGCACCGTGGAGAAGACAGTGGCCCCTACAGAATGTTCA

SEQ ID NO: 27 - protein, 642 aa, Artificial Sequence
  Synthetic Construct
TCCTATGAGCTGACCCAGCCACCCTCCGTGTCTGTGAGCCCAGGCCAGACCGCCAGGATCACATGTTCCGGCGATACACTGCCCAAGCGGAGCGGCTACTGGTATCAGCAGAAGCCAGACCAGGCCCCCCTGCTGGTCATCAACAAGGATACCGAGAGGCCTTCTGGCATCCCAGAGCGGTTCAGCGGCAGCAAATCTGGCACCACAGTGACCCTGACAATCAGCGGAGTGCAGGCAGAGGACGAGGCAGATTACTATTGCCAGTCCGCCGACAGCTCCTTCACCTACCCAAGCTTTGGCGGCGGCACCAAGCTGACAGTGCTGGGTCAGCCCAAGGCTGCCCCCTCGGTCACTCTGTTCCCGCCCTCGAGTGAGGAGCTTCAAGCCAACAAGGCCACACTGGTGTGTCTCATAAGTGACTTCTACCCGGGAGCCGTGACAGTGGCCTGGAAGGCAGATAGCAGCCCCGTCAAGGCGGGAGTGGAGACCACCACACCCTCCAAACAAAGCAACAACAAGTACGCGGCCAGCAGCTACCTGAGCCTGACGCCTGAGCAGTGGAAGTCCCACAGAAGCTACAGCTGCCAGGTCACGCATGAAGGGAGCACCGTGGAGAAGACAGTGGCCCCTACAGAATGTTCA

SEQ ID NO: 28 - protein, 1353 aa, Artificial Sequence
  Synthetic Construct
CAGGTGCAGCTGCAGGAGAGCGGACCTGGACTGGTGAAGCCATCCGGCACCCTGTCTCTGACCTGCACAGTGTCTGGCGGCTTCATCAGCTCCCGGAACTGGTGGAGCTGGGTGAGACAGCCACCTGGCAAGGGCCTGGAGTGGATCGGCGAGGTGTACCACACCGGCATCACAAACTATAACCCCTCCCTGAAGTCTCGGGTGACCATCAGCGTGGACAAGTCCAAGAATCAGTTCAGCCTGAAGCTGTCTAGCGTGACCGCCGCCGATACAGCCGTGTACTATTGCGCCAGGGAGGTGGTGGGACCTACCACAGGCTACTTTGACCTGTGGGGAAGGGGCACCCTGGTGACAATCTCCTCTGCGTCGACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCTGTGACGGTCTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAAGGTTGAGCCCAAATCTTGTGACAAAACTCACACATGCCCACCGTGCCCAGCACCTGAACTCCTGGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCCCCGGGTAAA

SEQ ID NO: 29 - protein, 10 aa, Artificial Sequence
  Synthetic Construct
  VARIANT (1)..(10): Xaa can be any naturally occurring amino acid
XQSAXFTYPX

SEQ ID NO: 30 - protein, 13 aa, Artificial Sequence
  Synthetic Construct
  VARIANT (1)..(13): Xaa can be any naturally occurring amino acid
XQSADSSFTYPSX

SEQ ID NO: 31 - protein, 9 aa, Artificial Sequence
  Synthetic Construct
GGFISSRNW

SEQ ID NO: 32 - protein, 9 aa, Artificial Sequence
  Synthetic Construct
  VARIANT (1)..(9): Xaa can be any naturally occurring amino acid
XWXEXIXNX

SEQ ID NO: 33 - protein, 9 aa, Artificial Sequence
  Synthetic Construct
  VARIANT (1)..(9): Xaa can be any naturally occurring amino acid
XWXEXITNX

SEQ ID NO: 34 - protein, 9 aa, Artificial Sequence
  Synthetic Construct
  VARIANT (1)..(9): Xaa can be any naturally occurring amino acid
XEXVXGYFX

SEQ ID NO: 35 - protein, 12 aa, Artificial Sequence
  Synthetic Construct
  VARIANT (1)..(12): Xaa can be any naturally occurring amino acid
XEXVGPXTGYFX

SEQ ID NO: 36 - protein, 32 aa, Artificial Sequence
  Synthetic Construct
GIPERFSGSSSGTTVTLTISGVQAEDEADYYC

SEQ ID NO: 37 - protein, 108 aa, Artificial Sequence
  Synthetic Construct
SYELTQPPSVSVSPGQTARITCSGDTLPKRSGYWYQQKPDQAPLLVINKDTERPSGIPERFSGSSSGTTVTLTISGVQAEDEADYYCQSADSSFTYPSFGGGTKLTVL

SEQ ID NO: 38 - protein, 214 aa, Artificial Sequence
  Synthetic Construct
SYELTQPPSVSVSPGQTARITCSGDTLPKRSGYWYQQKPDQAPLLVINKDTERPSGIPERFSGSSSGTTVTLTISGVQAEDEADYYCQSADSSFTYPSFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 32 aa, Artificial Sequence
  Synthetic Construct
GIPERFSGSNSGTTVTLTISGVQAEDEADYYC

SEQ ID NO: 40 - protein, 108 aa, Artificial Sequence
  Synthetic Construct
SYELTQPPSVSVSPGQTARITCSGDTLPKRSGYWYQQKPDQAPLLVINKDTERPSGIPERFSGSNSGTTVTLTISGVQAEDEADYYCQSADSSFTYPSFGGGTKLTVL

SEQ ID NO: 41 - protein, 214 aa, Artificial Sequence
  Synthetic Construct
SYELTQPPSVSVSPGQTARITCSGDTLPKRSGYWYQQKPDQAPLLVINKDTERPSGIPERFSGSNSGTTVTLTISGVQAEDEADYYCQSADSSFTYPSFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 32 aa, Artificial Sequence
  Synthetic Construct
GIPERFSGSTSGTTVTLTISGVQAEDEADYYC

SEQ ID NO: 43 - protein, 108 aa, Artificial Sequence
  Synthetic Construct
SYELTQPPSVSVSPGQTARITCSGDTLPKRSGYWYQQKPDQAPLLVINKDTERPSGIPERFSGSTSGTTVTLTISGVQAEDEADYYCQSADSSFTYPSFGGGTKLTVL

SEQ ID NO: 44 - protein, 214 aa, Artificial Sequence
  Synthetic Construct
SYELTQPPSVSVSPGQTARITCSGDTLPKRSGYWYQQKPDQAPLLVINKDTERPSGIPERFSGSTSGTTVTLTISGVQAEDEADYYCQSADSSFTYPSFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 45 - protein, 214 aa, Artificial Sequence
  Synthetic Construct
SYELTQPPSVSVSPGQTARITCSGDTLPKRSGYWYQQKPDQAPLLVINKDTERPSGIPERFSGSSSGTTVTLTISGVQAEDEADYYCQSADSSFTYPMFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - protein, 451 aa, Artificial Sequence
  Synthetic Construct
QVQLQESGPGLVKPSGTLSLTCTVSGGFISSRNWWSWVRQPPGKGLEWIGEVYHTGITNYIPSLKSRVTISVDKSKNQFSLKLSSVTAADTAVYYCAREVVGPTTGYFDLWGRGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 642 aa, Artificial Sequence
  Synthetic Construct
TCCTATGAGCTGACCCAGCCACCCTCCGTGTCTGTGAGCCCAGGCCAGACCGCCAGGATCACATGTTCCGGCGATACACTGCCCAAGCGGAGCGGCTACTGGTATCAGCAGAAGCCAGACCAGGCCCCCCTGCTGGTCATCAACAAGGATACCGAGAGGCCTTCTGGCATCCCAGAGCGGTTCAGCGGCAGCTCCTCTGGCACCACAGTGACCCTGACAATCAGCGGAGTGCAGGCAGAGGACGAGGCAGATTACTATTGCCAGTCCGCCGACAGCTCCTTCACCTACCCAATGTTTGGCGGCGGCACCAAGCTGACAGTGCTGGGTCAGCCCAAGGCTGCCCCCTCGGTCACTCTGTTCCCGCCCTCGAGTGAGGAGCTTCAAGCCAACAAGGCCACACTGGTGTGTCTCATAAGTGACTTCTACCCGGGAGCCGTGACAGTGGCCTGGAAGGCAGATAGCAGCCCCGTCAAGGCGGGAGTGGAGACCACCACACCCTCCAAACAAAGCAACAACAAGTACGCGGCCAGCAGCTACCTGAGCCTGACGCCTGAGCAGTGGAAGTCCCACAGAAGCTACAGCTGCCAGGTCACGCATGAAGGGAGCACCGTGGAGAAGACAGTGGCCCCTACAGAATGTTCA

SEQ ID NO: 48 - protein, 1353 aa, Artificial Sequence
  Synthetic Construct
CAGGTGCAGCTGCAGGAGAGCGGACCTGGACTGGTGAAGCCATCCGGCACCCTGTCTCTGACCTGCACAGTGTCTGGCGGCTTCATCAGCTCCCGGAACTGGTGGAGCTGGGTGAGACAGCCACCTGGCAAGGGCCTGGAGTGGATCGGCGAGGTGTACCACACCGGCATCACAAACTATATCCCCTCCCTGAAGTCTCGGGTGACCATCAGCGTGGACAAGTCCAAGAATCAGTTCAGCCTGAAGCTGTCTAGCGTGACCGCCGCCGATACAGCCGTGTACTATTGCGCCAGGGAGGTGGTGGGACCTACCACAGGCTACTTTGACCTGTGGGGAAGGGGCACCCTGGTGACAATCTCCTCTGCGTCGACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCTGTGACGGTCTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAAGGTTGAGCCCAAATCTTGTGACAAAACTCACACATGCCCACCGTGCCCAGCACCTGAACTCCTGGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCCCCGGGTAAA